SEQUENCE LISTING (27 sequences)

SEQ ID NO: 1 - DNA, 24 bp, artificial
  primer
ctccttgggttcgtctataaattg

SEQ ID NO: 2 - DNA, 19 bp, artificial
  primer
agtcagcagccagtctcag

SEQ ID NO: 3 - DNA, 24 bp, artificial
  primer
ctcggtggttagcaattattcctc

SEQ ID NO: 4 - DNA, 21 bp, artificial
  primer
agcccatagcattcgtctcag

SEQ ID NO: 5 - DNA, 20 bp, artificial
  primer
cttccatctgactcaggttt

SEQ ID NO: 6 - DNA, 21 bp, artificial
  primer
tgcttctgacggtatgtatta

SEQ ID NO: 7 - DNA, 24 bp, artificial
  primer
aatcagacagaacagttaatcctc

SEQ ID NO: 8 - DNA, 23 bp, artificial
  primer
tcaaccatatcttcaatgctacc

SEQ ID NO: 9 - DNA, 19 bp, artificial
  primer
taccaggactacgagattg

SEQ ID NO: 10 - DNA, 19 bp, artificial
  primer
tgccaggaaggtctattag

SEQ ID NO: 11 - DNA, 19 bp, artificial
  primer
catcatcttgctggttctg

SEQ ID NO: 12 - DNA, 20 bp, artificial
  primer
aggattgtaggtggatagtc

SEQ ID NO: 13 - DNA, 20 bp, artificial
  primer
ttggtcaggcagtataatcc

SEQ ID NO: 14 - DNA, 20 bp, artificial
  primer
gggcatatcctacaacaaac

SEQ ID NO: 15 - DNA, 1775 bp, Homo sapiens
gtaactgaaaatccacaagacagaatagccagatctcagaggagcctggctaagcaaaaccctgcagaacggctgcctaatttacagcaaccatgaggccacttaaggatgcagcaagaaggagccatctgcaatccaggaagaaattccttgccaggaaccaaattggttgtcaccttcatctaggacttctagcctcgagaacttacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaagaattatgaacgggccaaggcctgctttgaaaaggtgcttgaagtggaccctgaaaacccggaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaa

SEQ ID NO: 16 - DNA, 1714 bp, Homo sapiens
ggggcattttgtgcctgcctagctatccagacagagcagctaccctcagctctagctgatactacagacagtacaacagatcaagaagtatggcagtgacaactcgtttgacacggttgcacgaaaagatcctgcaaaatcattttggagggaagcggcttagccttctctataagggtagtgtccatggattccgtaatggagttttgcttgacagatgttgtaatcaagggcctactctaacagtgatttatagtgaagatcatattattggagcatatgcggaagagagttaccaggaaggaaagtatgcttccatcatcctttttgcacttcaagatactaaaatttcagaatggaaactaggactatgtacaccagaaacactgttttgttgtgatgttacaaaatataactccccaactaatttccagatagatggaagaaatagaaaagtgattatggacttaaagacaatggaaaatcttggacttgctcaaaattgtactatctctattcaggattatgaagtttttcgatgcgaagattcactggatgaaagaaagataaaaggggtcattgagctcaggaagagcttactgtctgccttgagaacttatgaaccatatggatccctggttcaacaaatacgaattctgctgctgggtccaattggagctgggaagtccagctttttcaactcagtgaggtctgttttccaagggcatgtaacgcatcaggctttggtgggcactaatacaactgggatatctgagaagtataggacatactctattagagacgggaaagatggcaaatacctgccgtttattctgtgtgactcactggggctgagtgagaaagaaggcggcctgtgcagggatgacatattctatatcttgaacggtaacattcgtgatagataccagtttaatcccatggaatcaatcaaattaaatcatcatgactacattgattccccatcgctgaaggacagaattcattgtgtggcatttgtatttgatgccagctctattcaatacttctcctctcagatgatagtaaagatcaaaagaattcgaagggagttggtaaacgctggtgtggtacatgtggctttgctcactcatgtggatagcatggatttgattacaaaaggtgaccttatagaaatagagagatgtgagcctgtgaggtccaagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaatagggaatctaagggaggaaattatcaactgtgcacaaggaaaaaaatagatatgtgaaaggttcacgtaaatttcctcacatcacagaagattaaaattcagaaaggagaaaacacagaccaaagagaagtatctaagaccaaagggatgtgttttattaatgtctaggatgaagaaatgcatagaacattgtagtacttgtaaataactagaaataacatgatttagtcataattgtgaaaaatagtaataatttttcttggatttatgttctgtatctgtgaaaaaataaatttcttataaaactcggaaaaaaaaaaaaaaaaaaaaaaaaaaaaa

SEQ ID NO: 17 - protein, 444 aa, Homo sapiens
MAVTTRLTRLHEKILQNHFGGKRLSLLYKGSVHGFRNGVLLDRCCNQGPTLTVIYSEDHIIGAYAEESYQEGKYASIILFALQDTKISEWKLGLCTPETLFCCDVTKYNSPTNFQIDGRNRKVIMDLKTMENLGLAQNCTISIQDYEVFRCEDSLDERKIKGVIELRKSLLSALRTYEPYGSLVQQIRILLLGPIGAGKSSFFNSVRSVFQGHVTHQALVGTNTTGISEKYRTYSIRDGKDGKYLPFILCDSLGLSEKEGGLCRDDIFYILNGNIRDRYQFNPMESIKLNHHDYIDSPSLKDRIHCVAFVFDASSIQYFSSQMIVKIKRIRRELVNAGVVHVALLTHVDSMDLITKGDLIEIERCEPVRSKLEEVQRKLGFALSDISVVSNYSSEWELDPVKDVLILSALRRMLWAADDFLEDLPFEQIGNLREEIINCAQGKK

SEQ ID NO: 18 - DNA, 2808 bp, Homo sapiens
gcggcggcggcggcgcagtttgctcatactttgtgacttgcggtcacagtggcattcagctccacacttggtagaaccacaggcacgacaagcatagaaacatcctaaacaatcttcatcgaggcatcgaggtccatcccaataaaaatcaggagaccctggctatcatagaccttagtcttcgctggtatactcgctgtctgtcaaccagcggttgactttttttaagccttcttttttctcttttaccagtttctggagcaaattcagtttgccttcctggatttgtaaattgtaatgacctcaaaactttagcagttcttccatctgactcaggtttgcttctctggcggtcttcagaatcaacatccacacttccgtgattatctgcgtgcattttggacaaagcttccaaccaggatacgggaagaagaaatggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttagagcccttctgaaaaagtatcctgcttctgatatgcagttttccttaaattatctaaaatctgctagggaatatcaatagatatttaccttttattttaatgtttcctttaattttttactatttttactaatctttctgcagaaacagaaaggttttcttctttttgcttcaaaaacattcttacattttactttttcctggctcatctctttattcttttttttttttttaaagacagagtctcgctctgttgcccaggctggagtgcaatgacacagtcttggctcactgcaacttctgcctcttgggttcaagtgattctcctgcctcagcctcctgagtagctggattacaggcatgtgccacccacccaactaatttttgtgtttttaataaagacagggtttcaccatgttggccaggctggtctcaaactcctgacctcaagtaatccacctgcctcggcctcccaaagtgctgggattacagggatgagccaccgcgcccagcctcatctctttgttctaaagatggaaaaaccacccccaaattttctttttatactattaatgaatcaatcaattcatatctatttattaaatttctaccgcttttaggccaaaaaaatgtaagatcgttctctgcctcacatagcttacaagccagctggagaaatatggtactcattaaaaaaaaaaaaaaagtgatgtacaacc

SEQ ID NO: 19 - protein, 551 aa, Homo sapiens
MAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSEETSVKSDYLSSGSFATTCESQSNSLVTSTLASESSSEGDFSADTSEINSNSDSLNSSSLLMNGLRNNQRKAKRSLAPRFDLPDMKETKYTVDKRFGMDFKEIELIGSGGFGQVFKAKHRIDGKTYVIKRVKYNNEKAEREVKALAKLDHVNIVHYNGCWDGFDYDPETSDDSLESSDYDPENSKNSSRSKTKCLFIQMEFCDKGTLEQWIEKRRGEKLDKVLALELFEQITKGVDYIHSKKLIHRDLKPSNIFLVDTKQVKIGDFGLVTSLKNDGKRTRSKGTLRYMSPEQISSQDYGKEVDLYALGLILAELLHVCDTAFETSKFFTDLRDGIISDIFDKKEKTLLQKLLSKKPEDRPNTSEILRTLTVWKKSPEKNERHTC

SEQ ID NO: 20 - DNA, 2058 bp, Homo sapiens
gcacgaggaagccacagatctcttaagaactttctgtctccaaaccgtggctgctcgataaatcagacagaacagttaatcctcaatttaagcctgatctaacccctagaaacagatatagaacaatggaagtgacaacaagattgacatggaatgatgaaaatcatctgcgcaactgcttggaaatgtttctttgagtcttctctataagtctagtgttcatggaggtagcattgaagatatggttgaaagatgcagccgtcagggatgtactataacaatggcttacattgattacaatatgattgtagcctttatgcttggaaattatattaatttacgtgaaagttctacagagccaaatgattccctatggttttcacttcaaaagaaaaatgacaccactgaaatagaaactttactcttaaatacagcaccaaaaattattgatgagcaactggtgtgtcgtttatcgaaaacggatattttcattatatgtcgagataataaaatttatctagataaaatgataacaagaaacttgaaactaaggttttatggccaccgtcagtatttggaatgtgaagtttttcgagttgaaggaattaaggataacctagacgacataaagaggataattaaagccagagagcacagaaataggcttctagcagacatcagagactataggccctatgcagacttggtttcagaaattcgtattcttttggtgggtccagttgggtctggaaagtccagttttttcaattcagtcaagtctatttttcatggccatgtgactggccaagccgtagtggggtctgataccaccagcataaccgagcggtataggatatattctgttaaagatggaaaaaatggaaaatctctgccatttatgttgtgtgacactatggggctagatggggcagaaggagcaggactgtgcatggatgacattccccacatcttaaaaggttgtatgccagacagatatcagtttaattcccgtaaaccaattacacctgagcattctacttttatcacctctccatctctgaaggacaggattcactgtgtggcttatgtcttagacatcaactctattgacaatctctactctaaaatgttggcaaaagtgaagcaagttcacaaagaagtattaaactgtggtatagcatatgtggccttgcttactaaagtggatgattgcagtgaggttcttcaagacaactttttaaacatgagtagatctatgacttctcaaagccgggtcatgaatgtccataaaatgctaggcattcctatttccaatattttgatggttggaaattatgcttcagatttggaactggaccccatgaaggatattctcatcctctctgcactgaggcagatgctgcgggctgcagatgattttttagaagatttgcctcttgaggaaactggtgcaattgagagagcgttacagccctgcatttgagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagcacagtgcctggtaatgagcaagcatacttgccattacttttccttcccactctctccaacatcacattcactttaaatttttctgtatatagaaaggaaaactagcctgggcaacatgatgaaaccccatctccactgc

SEQ ID NO: 21 - protein, 413 aa, Homo sapiens
MVERCSRQGCTITMAYIDYNMIVAFMLGNYINLRESSTEPNDSLWFSLQKKNDTTEIETLLLNTAPKIIDEQLVCRLSKTDIFIICRDNKIYLDKMITRNLKLRFYGHRQYLECEVFRVEGIKDNLDDIKRIIKAREHRNRLLADIRDYRPYADLVSEIRILLVGPVGSGKSSFFNSVKSIFHGHVTGQAVVGSDTTSITERYRIYSVKDGKNGKSLPFMLCDTMGLDGAEGAGLCMDDIPHILKGCMPDRYQFNSRKPITPEHSTFITSPSLKDRIHCVAYVLDINSIDNLYSKMLAKVKQVHKEVLNCGIAYVALLTKVDDCSEVLQDNFLNMSRSMTSQSRVMNVHKMLGIPISNILMVGNYASDLELDPMKDILILSALRQMLRAADDFLEDLPLEETGAIERALQPCI

SEQ ID NO: 22 - DNA, 2787 bp, Homo sapiens
agagcggaggccgcactccagcactgcgcagggaccgccttggaccgcagttgccggccaggaatcccagtgtcacggtggacacgcctccctcgcgcccttgccgcccacctgctcacccagctcaggggctttggaattctgtggccacactgcgaggagatcggttctgggtcggaggctacaggaagactcccactccctgaaatctggagtgaagaacgccgccatccagccaccattccaaggaggtgcaggagaacagctctgtgataccatttaacttgttgacattacttttatttgaaggaacgtatattagagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacatcgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaaattaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcag

SEQ ID NO: 23 - protein, 662 aa, Homo sapiens
MVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKINAFNQDITALMQGEETVGEEDIRLFTRLRHEFHKWSTIIENNFQEGHKILSRKIQKFENQYRGRELPGFVNYRTFETIVKQQIKALEEPAVDMLHTVTDMVRLAFTDVSIKNFEEFFNLHRTAKSKIEDIRAEQEREGEKLIRLHFQMEQIVYCQDQVYRGALQKVREKELEEEKKKKSWDFGAFQSSSATDSSMEEIFQHLMAYHQEASKRISSHIPLIIQFFMLQTYGQQLQKAMLQLLQDKDTYSWLLKERSDTSDKRKFLKERLARLTQARRRLAQFPG

SEQ ID NO: 24 - DNA, 2545 bp, Homo sapiens
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 25 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 26 - DNA, 1331 bp, Homo sapiens
tcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaattt

SEQ ID NO: 27 - protein, 218 aa, Homo sapiens
MATRSPGVVISDDEPGYDLDLFCIPNHYAEDLERVFIPHGLIMDRTERLARDVMKEMGGHHIVALCVLKGGYKFFADLLDYIKALNRNSDRSIPMTVDFIRLKSYCNDQSTGDIKVIGGDDLSTLTGKNVLIVEDIIDTGKTMQTLLSLVRQYNPKMVKVASLLVKRTPRSVGYKPDFVGFEIPDKFVVGYALDYNEYFRDLNHVCVISETGKAKYKA

(19) United States
(12) Patent Application Publication
(10) Pub. No.: US 2007/0117105 A1
(43) Pub. Date: May 24, 2007

(54) Interferon assay

(76) Inventors: Mary K. Crow, New York, NY (US); Kyriakos Kirou, Astoria, NY (US); Jing Hua, Elmhurst, NY (US)

Correspondence Address: DARBY & DARBY P.C., P. O. BOX 5257, NEW YORK, NY, 10150-5257, US

(21) Appl. No.: 11/431,775

(22) Filed: May 9, 2006

Related U.S. Application Data
(60) Provisional application No. 60/680,931, filed on May 12, 2005.

(51) Int. Cl. C12Q 1/68; G01N 33/567

(52) U.S. Cl. 435/6; 435/7.2

(57) ABSTRACT

The present invention relates to methods and reporter cell assays for determining the ability of a patient sample to induce interferon target gene expression in interferon responsive cells. These methods will be useful for detecting, diagnosing, and monitoring those who have or are at risk of various autoimmune disorders or diseases, including systemic lupus erythematosus (SLE) and Sjogren's syndrome.

### CROSS REFERENCE TO RELATED APPLICATIONS

This application claims priority from U.S. Provisional Application Ser. No. 60/680,931, filed May 12, 2005, which is hereby incorporated by reference in its entirety.

### FEDERALLY SPONSORED RESEARCH OR DEVELOPMENT

This invention was made in part in the course of research sponsored by the National Institutes of Health (NIH) Grants AR-050829 and AI-052422, the Alliance for Lupus Research, the Lupus Research Institute, and the Mary Kirkland Center for Lupus Research. The U.S. government may have certain rights in this invention.

### FIELD OF THE INVENTION

The present invention relates to methods and reporter cell assays for determining the ability of a patient sample to induce interferon target gene expression in interferon responsive cells.

### BACKGROUND OF THE INVENTION

Prototypical systemic autoimmune diseases include systemic lupus erythematosus (SLE), scleroderma, mixed connective tissue disease, Sjogren's syndrome and other systemic disorders. Epidemiological studies indicate that typical onset of these diseases occurs in the teenage years to the 20's (i.e., post-puberty). Additionally, studies indicate that these diseases affect women in significantly greater numbers than men, in a ratio of about 8-9:1.

These disease states are characterized by generalized immune system activation, but with evidence for antigen-specific induction of T cell-dependent autoantibodies. For example, in SLE autoantibody specificities are very characteristic. Autoantigens include nucleosomes (particles containing histones and DNA); ribonucleoprotein (RNP) particles (containing RNA and proteins that mediate specialized functions in the RNP particle); and double stranded DNA. An example of proteins that mediate specialized functions in the RNP particles is the Sm protein, which has spliceosome function. It is theorized that an inappropriate immune response is initiated in SLE. The response appears to be initiated to a component of one of the intracellular particles, which then spreads to other components of the particle. Tissue damage mostly occurs through actions of the autoantibodies, including activation of the complement system, although antigen-specific T cells also may play a direct role in tissue damage. The tissue damage may also be triggered or exacerbated by drugs that may demethylate DNA and by sunlight (e.g. UV light).

Organ targeted autoimmune diseases include IDDM, MS, autoimmune thyroid disease, rheumatoid arthritis (RA), pemphigus, psoriasis, polymyositis, dermatomyositis, pemphigoid, vitiligo, primary bilary cirrhosis, chronic active hepatitis, Crohn's disease, ulcerative colitis and pernicious anemia. Epidemiological studies indicate that these diseases have variable onset. Gender distribution studies indicate that some are more common in females, whereas others have a more even gender distribution.

These disease states are also characterized by an inappropriate immune response to a self-protein. The response often spreads to include other antigens, notably those that are enriched in the target organ. For example in IDDM, the earliest detectable immune response is directed at the protein glutamic acid decarboxylase (GAD) with later responses directed toward insulin. The self-proteins targeted in some other organ-specific autoimmune diseases include, desmoglein 3 in pemphigus vulgaris, desmoglein 1 in pemphigus foliaceus, myelin oligodendrocyte glycoprotein in MS, tyrosinase related protein in vitiligo, thyroid stimulating hormone receptor in autoimmune thyroid disease, bullous pemphigoid antigen 1 in bullous pemphigoid, and SP100 in primary bilary cirrhosis. There are other complex autoimmune diseases, rheumatoid arthritis for example, in which the relevant autoantigens have not been identified. Antigen-specific T-cells triggered by these antigens mediate tissue damage in the target organ. Cytokines and autoantibodies also may contribute to development of the disease state.

SLE is a specific example of a chronic inflammatory disease that can affect various parts of the body including skin, blood, kidney, and joint. In some patients, SLE may be a mild disease, however, in other patients is may be a serious and life-threatening disease. More than 16,000 cases of SLE are reported in the United States each year, with up to 1.5 million cases diagnosed. Although SLE can occur at any age, and in either sex, it has been found to occur 1-15 times more frequently in women.

In SLE immune complexes may be deposited throughout the body including in the glomeruli, skin, lungs, synovium and mesothelium. Renal disease is a common consequence of SLE. Physical manifestations of SLE include skin rashes, typically across the cheek or jaw regions, effusion in body cavities, including pericardial effusions, pericarditis, endocarditis, arthralgia and renal failure. However, there may be stages of the disease when few symptoms are evident, and patients with SLE may not necessarily exhibit identical symptoms. Some symptoms mimic other illnesses. In view of the complexities of autoimmune disease symptoms in general, and for lupus in particular, these diseases are difficult to diagnose.

To date there is no single laboratory test that can definitively detect lupus. The lupus erythematosus cell test is not specific for SLE. The immuno-fluorescent antinuclear antibody (ANA) test is more sensitive, however, positive results are inconclusive because they may be indicative of other diseases. Skin and kidney biopsies may also be performed in an attempt to diagnose SLE, but these are invasive, costly and often not definitive. Recent methods for detecting SLE involve determining the expression profile of specific genes from patient samples. However, these tests have the drawback of detecting only specifically known genes and certain methods require the use of fresh patient cells. Other similar methods involve using nucleic acid microarrays to determine gene expression profiles in SLE patient cells and involve extensive interpretation of the expression profile results.

Additionally, there are currently no good biomarkers for monitoring disease progression in patients with SLE. In some instances the titer of anti-double stranded DNA antibodies can be used to monitor disease state or progression in certain patients; however, this method has limited application and is difficult to interpret.

The difficulties associated with diagnosing and monitoring SLE demonstrate a need in the art for methods for more accurately determining whether a patient has SLE, for monitoring progression of the disease and response to therapy, or for determining if a patient is predisposed to having SLE. There is also a need in the art for methods to identify patients with particular manifestations of disease that are indicative of disease severity or disease state. Additionally, there is a need in the art for methods of identifying patients for inclusion in interventional clinical trials, i.e. those that are likely to respond to the test agent, and for monitoring patient response to therapy in interventional trials. These difficulties are also generally associated with diagnosing systemic autoimmune diseases and organ targeted immune diseases and demonstrate a need in the art for methods for more accurately diagnosing, monitoring, and determining if a patient is predisposed to having a systemic or organ targeted autoimmune disease.

### SUMMARY OF THE INVENTION

The present invention provides methods for detecting mediators that stimulate expression of interferon-inducible genes in a subject; methods for diagnosing systemic lupus erythematosus (SLE) in a subject; methods for monitoring a subject with SLE; and methods for assessing the efficacy of a test compound in treating SLE.

In certain embodiments, the present invention provides methods for detecting mediators that stimulate expression of interferon-inducible genes in a subject by:
a) contacting interferon responsive cells in vitro with a body fluid sample obtained from the subject, wherein the interferon responsive cells are not the subject's cells;
b) detecting the expression level of at least one interferon-inducible gene (IFIG) by the interferon responsive cells of step a);
c) detecting the expression level of at least one interferon-inducible gene (IFIG) by control interferon responsive cells, wherein the control interferon responsive cells are not the subject's cells; and
d) comparing the expression level detected in step b) with the expression level detected in step c), wherein an increased expression level in step b) is indicative that the subject has an elevated level of mediators that stimulate expression of interferon-inducible genes, and the elevated level is indicative of an autoimmune disorder or disease.

In certain embodiments, the invention includes methods for diagnosing systemic lupus erythematosus (SLE) in a subject by:
a) contacting interferon responsive cells in vitro with a body fluid sample obtained from the subject, wherein the interferon responsive cells are not the subject's cells;
b) detecting the expression level of at least one interferon-inducible gene (IFIG) by the interferon responsive cells of step a);
c) detecting the expression level of at least one interferon-inducible gene (IFIG) by control interferon responsive cells, wherein the control interferon responsive cells are not the subject's cells; and
d) comparing the expression level detected in step b) with the expression level detected in step c), wherein an increased expression level detected in step b) as compared with the expression level detected in step c) is indicative that the subject has SLE.

Certain methods include contacting the control interferon responsive cells of step c) with a body fluid sample obtained from a healthy subject, prior to detecting. Other methods involve contacting the control interferon responsive cells of step c) with media, prior to detecting.

A further embodiment of the invention includes methods for monitoring a subject with systemic lupus erythematosus (SLE) by:
a) contacting interferon responsive cells in vitro with a body fluid sample obtained from a subject prior to administration of a test compound, wherein the interferon responsive cells are not the subject's cells;
b) contacting interferon responsive cells in vitro with a body fluid sample obtained from a subject at one or more time points following administration of a test compound, wherein the interferon responsive cells are not the subject's cells;
c) detecting the expression level of at least one interferon-inducible gene by the interferon responsive cells of step a);
d) detecting the expression level of at least one interferon-inducible gene by the interferon responsive cells of step b); and
e) comparing the expression level of step c) with the expression level of step d), wherein a decreased expression level of at least one interferon-inducible gene by the cells of step d) as compared to the expression level of step c) is indicative of efficacy of the test compound.

Yet another embodiment of the present invention includes a method for assessing the efficacy of a test compound in treating systemic lupus erythematosus (SLE) by:
a) contacting interferon responsive cells in vitro with a body fluid sample obtained from a subject prior to administration of a test compound, wherein the interferon responsive cells are not the subject's cells;
b) contacting interferon responsive cells in vitro with a body fluid sample obtained from a subject at one or more time points following administration of a test compound, wherein the interferon responsive cells are not the subject's cells; and
c) detecting the expression level of at least one interferon-inducible gene by the interferon responsive cells of step a);
d) detecting the expression level of at least one interferon-inducible gene by the interferon responsive cells of step b); and
   e) comparing the expression level of step c) with the expression level of step d), wherein a decreased expression level of at least one interferon-inducible gene by the cells of step d) as compared to the expression level of step c) is indicative of efficacy of the test compound.

In some methods the interferon responsive cells are selected from the group consisting of A-549 cells, AG1732 cells, HeLa cells, HepG2 cells, Hep-2 cells, Huh-7 cells, G-361 cells, and WISH cells. In some methods the interferon responsive cells are WISH cells.

In some methods the detecting steps are carried out using real-time quantitative PCR.

In some methods the interferon-inducible genes (IFIG) are interferon-α inducible genes. In some methods the interferon-αinducible genes are selected from the group consisting of IFI44 (SEQ ID NO:16), C1orf29 (SEQ ID NO:20), PRKR (SEQ ID NO:18), IFIT1 (SEQ ID NO:15), and MX1 (SEQ ID NO:22).

In some methods the body fluid sample is plasma or serum.

### BRIEF DESCRIPTION OF THE DRAWINGS

FIGS. 1A and 1B show time course and dose response of IFNα target gene expression induced by rhIFNα or SLE plasma.

FIGS. 2A-2E show SLE plasma, but not RA or healthy donor plasma, induces IFNα target gene expression.

FIGS. 3A-3E show that SLE plasma activity correlates with IFNα score in PBMC cells.

FIGS. 4A-4D show inhibition of SLE plasma activity in cell system by polyclonal and monoclonal anti-IFNα antibodies.

FIGS. 5A-5D show IFNα activity in SLE plasma correlates with IFNα score in PBMC cell system and anti-RNA binding protein autoantibody titer.

### DETAILED DESCRIPTION

The present invention relates to assays, i.e. reporter cell assays for detecting the ability of a patient body fluid sample to induce interferon target gene expression in interferon responsive cells, wherein the interferon responsive cells are not the subject's cells. This assay will be useful for assessing disease activity, response to therapy, as well as efficacy of potential therapeutics that target the interferon pathway. It will also be useful in research studies to characterize the components of the factors that induce interferon pathway activation. The interferon responsive gene expression assay of the present invention can sensitively and reliably detect interferon (IFN) activity in SLE plasma and serum.

In certain embodiments, the present invention provides methods for determining the ability of mediators in a body fluid, such as serum or plasma, to stimulate expression of interferon-inducible genes in IFN responsive cells, such as WISH cells. The assays of the present invention do not utilize patient cells as the IFN responsive cells. The expression of the interferon-inducible genes (IFIG) can be tested or verified by measuring the levels of RNA in the WISH cell exposed to the patient or control sample, followed by RT-PCR and real time PCR and comparing the results to a control WISH cell that has been exposed to a healthy patient sample, or a control cell that has not been exposed or contacted with the patient sample.

Suitable cells for the present in vitro assays include any suitable cell lines that are IFN responsive, that is cells that are capable of binding to IFN in a manner reflective of the induction of interferon inducible genes. Examples of such cells include: WISH cells (American Type Culture Collection, reference CCL-25, Manassas, Va.), A-549 lung carcinoma cells (A-549 cells, ATCC reference CCL-185), G-361 human melanoma cells (G-361 cells, ATCC reference, CRL-1424), Hep-2 human laryngeal carcinoma cells (Hep-2 cells, ATCC reference CCL-23), human hepatocellular carcinoma cell line HepG2 (HepG2 cells, ATCC reference HB-8065), Huh-7 cell lines, AG1732 human cell line cells, primary human hepatocytes, and HeLa cells. Additionally, bovine kidney MDBK cells (MDBK cells, ATCC reference CCL-22), BT bovine cells (ATCC reference CRL-1390), guinea pig embryo cells (such as ATCC reference CCL-242), and SPEV pig cell line cells will be suitable in assays of the present invention (37, 41, 42). Many of these cell lines are described in the 2003/2004 Edition of the ATCC Catalog of Cell Lines & Hybridomas, American Type Culture Collection, Rockville, Md.

Interferons (IFN) are multi-functional proteins that induce a large number of genes which mediate many biological processes including host defense, cell growth control, signaling, and metabolism. Human epithelial cell lines such as WISH cells are especially responsive to type I IFN (39). A549 lung epithelial cells have been shown to be responsive to IFNα in studies that showed treatment with IFNα or TNF-α enhanced expression of RIG-I, IKKepsilon, interferon regulatory factor (IRF)1, IRF7 and p50 proteins (38). Studies have shown that HeLa cells, AG1732 cells, MDBK cells, and BT cells are responsive to human rIFNα A (37). The studies showed using WISH cell activity response to human rIFNα A as 100% activity, that AG1732 cells exhibited 90% activity, HeLa cells exhibited 94% activity, MDBK and BT cells exhibited 170-190% activity to rIFNα A. (37). Studies have also shown a response to IFNα stimulation in HepG2, Huh-7, and primary human hepatocytes, indicated by up-regulation of APOBEC3G (40). Likewise, guinea pig cells showed 182% activity to recombinant human IFNα A, rIFN-α2 and lymphoblastoid IFN as compared to WISH cell activity (41). SPEV pig cells and MDBK calf cells were responsive to human interferon omega (IFN-Ω) (42).

Previously, WISH cells have been used in cytopathic assays for detecting IFN-mediated inhibition of viral infection of the cells. The presence of IFN was determined by the decrease in viral plaques on IFN treated WISH cells. In contrast, the present assay method measures interferon target gene expression in cells responsive to IFN, such as WISH cells, or other suitable cells, by RT-PCR followed by real time PCR, instead of measuring the effect of interferon on cell death induced by viral infection.

In certain embodiments, a WISH cell assay is used to measure the functional effects of patient plasma or serum components on the gene expression of WISH cells. It was found that IFNα-regulated genes were induced by SLE plasma samples, but not by most of the RA or healthy control plasma samples. The activity in SLE plasma was inhibited by anti-IFNα antibody, but not by anti-IFNβ or γ antibodies. It was concluded that IFNα in SLE plasma is a stimulus of IFN target gene expression and is related to expression of those genes in PBMCs from SLE patients and to the titer of anti-RNA binding protein (RBP) autoantibodies. The present data provides support that IFNα mediates immune system activation and dysregulation in SLE.

In certain embodiments, WISH cells, which have a large amount of interferon receptors, are contacted with plasma (or other fluid) from a subject, and interferon-inducible genes (IFIG) in the contacted WISH cells are then quantified by real-time PCR. The assay measures the capacity of the plasma to interact with the cells to “turn on” target genes. Other cell types that have IFN receptors, as described above may also be used as IFN responsive cells in the in vitro assays of the present invention.

In certain embodiments, a WISH cell assay is used to measure the functional effects of patient plasma or serum components on the gene expression of WISH cells. It was found that certain IFIGs, such as IFNα-regulated genes were induced by SLE plasma samples, but not by healthy control plasma samples or by most of the rheumatoid arthritis (RA) plasma samples. Various type I IFN inducible genes could be used as the target genes in the present assays. Non-limiting examples of these genes have been described and include C1orf29, myxovirus (influenza virus) resistance 1 (MX1), interferon-induced protein with tetratricopeptide repeats 1 (IFIT1), interferon-induced protein 44 (IFI44), protein kinase, interferon-inducible double stranded RNA dependent (PRKR), 2′-5′ oligoadenylate synthetase 3 (OAS3), guanylate nucleotide binding protein 1 (GBP1), human interferon regulatory factor-1 (IRF1), serpin peptidase inhibitor (SERPING1), chemokine (CXC motif) ligand 9 (CXCL9), chemokine (CXC motif) ligand 10 (CXCL10), proteasome subunit, beta type, 8 (PSMB8), proteasome subunit, beta type, 10 (PSMB10), G protein-coupled receptor 105 (GPR105), Fc fragment of IgG, high affinity Ia, receptor (CD64) (FCGR1A) (10).

Thus, in certain embodiments, the present invention provides methods for contacting interferon responsive cells, such as WISH cells, with a body fluid sample from a subject and detecting the expression level of at least one interferon-inducible gene. This level of expression (resulting from the exposure of the WISH cells to the subject's sample) will be compared with the level of expression of the IFIGs, such as IFNα-regulated genes, in WISH cells contacted with a body fluid sample from a healthy subject or patient.

In other embodiments, the level of expression resulting from the exposure of the WISH cells to the subject's sample will be compared with a standard, or predetermined level of expression of the IFIGs, such as IFNα-regulated genes, in control WISH cells. In certain embodiments, the control WISH cells include those treated with media or other nutritional components that do not induce expression of IFIG genes. In other embodiments, the control WISH cells include those treated with a sample from a healthy subject or patient. Samples from healthy subjects or patients (those patients or subjects that do not have an autoimmune disease) do not induce expression of IFIG genes in IFN responsive cells, such as WISH cells. Serum or plasma samples from SLE patients induce the expression of certain IFIGs, such as IFNα-regulated genes, in WISH cells. Thus, comparing the level of expression of the IFIGs induced by WISH cells contacted with a test subject's sample, with the level of expression of IFIGs induced by WISH cells contacted with a healthy subject's sample, indicates a subject is likely to have a systemic autoimmune disease such as SLE or Sjogren's syndrome, if the comparison shows a greater level of expression of the IFIGs by the subject's sample than by the healthy sample and the increased IFIG expression correlates with the autoimmune disease. The level of expression of IFIGs can vary from weak expression to strong expression. The magnitude of the expression level of IFIGs will correlate with the disease state, with weakly expressed IFIGs indicating a predisposition to disease or a mild disease state, while strongly expressed IFIGs indicating active disease state.

Advantages of assays of the present invention include that stored serum samples and commercially available IFN responsive cells, as described herein such as WISH cells, A-549 cells, G-361 cells, or Hep-2 cells, may be used. Fresh cells and fresh samples, while suitable for use, are not required for the present assays.

“Systemic lupus erythematosus” as used herein refers to the physiological status of an individual that may have or develop systemic lupus erythematosus (SLE), as reflected in one or more markers or indicators including genotype. Assessing the absence or presence of a pathology or symptom of SLE may be useful in determining the individual's predisposition to developing such a condition. SLE disease markers include, without limitation, clinical measurements, such as, e.g. skin rashes, typically across the cheek or jaw regions, effusion in body cavities, including pericardial effusions, pericarditis, endocarditis, arthralgia, and renal failure. These clinical indications of SLE are assessed using conventional methods well known in the art. Also included in the evaluation of SLE status are quantitative or qualitative changes in certain markers with time, such as would be used, e.g., in the determination of an individual's response to a particular therapeutic regimen or of a predisposed individual's eventual development of SLE.

It will be understood that a diagnosis of SLE made by a medical practitioner encompasses not only clinical measurements but also medical judgment.

A “predisposition to develop systemic lupus erythematosus” refers to an increased likelihood, relative to the general population, to develop SLE, as defined above. A predisposition does not signify certainty, and development of the disease may be forestalled or prevented by prophylaxis, e.g., adopting a modified diet, exercise program, or treatment with gene therapy or pharmaceuticals. An advantage of the present invention is that it permits identification of individuals, based on the results of methods using a patient sample to stimulate expression of interferon-inducible genes in IFN responsive cells such as WISH cells, who are predisposed to develop SLE, and for whom prophylactic intervention can be especially important.

An “interferon responsive cell” is any suitable cell available for cell culture, that is IFN responsive, that is cells that are capable of binding to IFN in a manner reflective of the induction of interferon inducible genes. The interferon responsive cells used in the present methods are not the subject's cells. Suitable cells for the present assay include cells such as: WISH cells (American Type Culture Collection, reference CCL-25, Manassas, Va.), A-549 lung carcinoma cells (A-549 cells, ATCC reference CCL-185), G-361 human melanoma cells (G-361 cells, ATCC reference, CRL-1424), Hep-2 laryngeal carcinoma cells (Hep-2 cells, ATCC reference CCL-23), HepG2 cells, Huh-7 cell lines, AG1732 human cell line cells, primary human hepatocytes, and HeLa cells. Additionally, MDBK calf cells, BT bovine cells, guinea pig embryo cells, and SPEV pig cell line cells will be suitable in assays of the present invention (37, 41, 42).

A “control”, “control value” or “reference value” in an assay is a value used to detect an alteration in, e.g., transcriptional activity of a gene, levels of a protein or mRNA detected in a sample taken from a patient or measured in a reconstituted system, or any other assays described herein. For instance, the presence of mediators in a patient sample, such as serum or plasma that stimulate expression of interferon-inducible genes in IFN responsive cells such as WISH cells, can be tested or verified by measuring the levels of RNA in the WISH cell followed by RT-PCR and real time PCR and comparing the results to a control WISH cell that has not been exposed or contacted with the patient sample. In addition, modulation, i.e., up- or down-regulation, of the transcriptional activity the inhibitory/stimulatory effect of an agent (e.g., a test compound) on modulation of a WISH cell exposed to a patient sample can be evaluated by comparing the measured value of transcriptional activity to that of a control value. The control or reference value may be, e.g., a predetermined reference value, or may be determined experimentally. For example, in such an assay, a control or reference may be, e.g., the transcriptional activity of a gene in the absence of an agent or test compound (for comparison with transcriptional activity in the presence of the agent); or any other suitable control or reference. In a diagnostic assay, a reference or control value may be obtained by comparing e.g., a nucleotide sequence, or a nucleotide or protein level measured, in a sample taken from a patient predisposed to or suspected of suffering from, a disease, to a corresponding sequence or measured value of a sample taken from a healthy, or “control” individual. A test compound may be any compound that may inhibit the production of or block the patient mediators that stimulate expression of interferon-inducible genes in IFN responsive cells such as WISH cells.

A “sample” refers to a biological material which can be tested for the presence of mediators that stimulate the expression of interferon-inducible genes. Such samples can be obtained from subjects, such as humans and non-human animals, and include blood and blood products, synovial fluid, tissue, especially glands, biopsies, plural effusions, cerebrospinal fluid (CSF), ascites fluid, and cell culture.

Cells, tissues, and fluids useful in the methods of this invention include whole blood, plasma, serum, urine, nasal secretions, synovial fluid, ocular secretions, vaginal secretions, and saliva. In certain embodiments blood, plasma, or serum is utilized in the practice of this invention.

When the sample is blood, methods may include processing the blood by a means known to the art, such as filtration or centrifugation, for separating plasma or serum which is to be assayed. As used herein, “whole blood” refers to blood as drawn. Whole blood contains a substantial amount of cells. As used herein, “plasma” refers to blood with no substantial amount of cells. Plasma does contain clotting factors. As used herein, “serum” refers to blood without a substantial amount of cells or clotting factors.

The term “cDNA” refers to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred.

In accordance with the present invention there may be employed conventional molecular biology, microbiology, cell biology, and recombinant DNA techniques within the skill of the art. Such techniques are explained fully in the literature. See e.g. Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Freshney (2005) Culture of Animal Cells: A Manual of Basic Technique. Wiley-Liss; 5 edition (Jul. 29, 2005); Coligan et al. eds. (2005) Current Protocols in Immunology, John Wiley and Sons, Inc.: Hoboken, N.J.

In certain embodiments, the present invention may utilize DNA segments that are complementary, or essentially complementary, to the sequences described herein. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a specified nucleic acid segment, under relatively stringent conditions such as those described herein below. Such sequences may encode a complete protein product or a functional or non-functional fragment thereof.

Similarly, any reference to a nucleic acid may be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the expression or detectability of IFN transcripts, IFN polypeptides, or desired fragments of IFN transcripts or IFN polypeptides.

Hybridizing nucleic acid segments may be relatively short nucleic acids, often termed oligonucleotides. Sequences of at least 10 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides are contemplated as well. Such oligonucleotides will find use, for example, as probes in Southern and Northern blots and as primers in amplification reactions.

Suitable nucleic acid hybridization conditions will be well known to those of skill in the art. Accordingly, the nucleotide sequences of the invention may be used for their ability to selectively form duplex molecules with complementary stretches of DNA fragments. Depending on the application envisioned, one will desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by about 0.02 M to about 0.15 M NaCl at temperatures of about 50° C. to about 70° C. Such selective conditions tolerate little, if any, mismatch between the probe and the template or target strand, and would be particularly suitable for isolating crystal protein-encoding DNA segments. Detection of DNA segments via hybridization is well-known to those of skill in the art, and the teachings of U.S. Pat. Nos. 4,965,188 and 5,176,995 (each specifically incorporated herein by reference) are exemplary of the methods of hybridization analyses.

In certain applications, for example, substitution of amino acids by site-directed mutagenesis, it is appreciated that lower stringency conditions are required. Under these conditions, hybridization may occur even though the sequences of probe and target strand are not perfectly complementary, with mismatches at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions include utilizing approximately 10 mM Tris-HCl (pH 8.3), 50 mM KCl, 1.5 μM MgCl₂, at temperatures ranging from approximately 40° C. to about 72° C. Formamide and sodium dodecyl sulfate (SDS) also may be used to alter the hybridization conditions.

In certain embodiments, it will be advantageous to employ nucleic acid sequences (e.g. primers or probes) utilized in the present invention in combination with an appropriate means, such as a label, for determining hybridization. A wide variety of appropriate indicator means are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmental undesirable reagents. In the case of enzyme tags, colorimetric indicator substrates are known that can be employed to provide a means visible to the human eye or spectrophotometrically, to identify specific hybridization with complementary nucleic acid-containing samples.

In general, it is envisioned that the hybridization probes described herein will be useful both as reagents in solution hybridization as well as in embodiments employing a solid phase. In embodiments involving a solid phase, the test DNA (or RNA) is adsorbed or otherwise affixed to a selected matrix or surface. This fixed, single-stranded nucleic acid is then subjected to specific hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface so as to remove nonspecifically bound probe molecules, specific hybridization is detected, or even quantitated, by means of the label.

### Experimental Design and Rationale

The IFN family includes several type I isoforms in addition to IFNα, including IFNβ, -κ, -τ, and -ω), as well as type III IFN (IFNλ) and type II IFN (IFNγ) (15,16,17). Although IFNα is the dominant type I IFN present in the setting of viral infections, it is possible that other isoforms are active in SLE. In addition, recent efforts to determine the factors that induce IFN in SLE have supported a role for immune complexes containing either DNA or RNA (18-23). Interaction of nucleic acid components of immune complexes with Toll-like receptors (TLRs) or RNA helicases could induce transcription of type I IFNs, but might also directly induce expression of some IFN target genes (24-26).

To characterize the mediators mainly responsible for activation of the IFN pathway in SLE, an assay was developed that measures the functional effects of plasma or serum components (mediators) on the gene expression of cultured target cells. These target cells are interferon responsive cells that can be used to detect the expression level of one or more interferon-inducible genes. The data described herein indicate that IFNα, rather than IFNβ or IFNγ, is present in many SLE plasmas. Moreover, the IFN activity measured in lupus plasmas shows a correlation with the expression of mRNAs encoded by interferon-inducible genes (IFIG) quantified in the same patients' PBMC and a strong correlation with the titer of autoantibodies specific for RNA-binding proteins present in those patients. Taken together, these data support a functional link between IFNα activity in lupus plasma, activation of IFIG in lupus PBMC, and targeting of the lupus immune response to particles containing small RNAs and RNA-binding proteins.

Patients and controls. Seventy-three SLE patients, nineteen rheumatoid arthritis (RA) patients and thirty healthy donors were studied. Forty-eight SLE patients, nineteen patients with RA, and twenty-eight healthy donors provided blood samples for the initial phase of the study. These donors of plasma samples were the same SLE patients, RA patients and healthy donors who had previously been studied for IFIG expression in PBMC (10,11) (allowing for relevant comparisons to be made as described below). Samples were obtained from some additional donors not previously studied, including twenty-five with SLE and two healthy subjects. SLE and RA patients were followed at the Hospital for Special Surgery and met the American College of Rheumatology classification criteria for SLE or RA (27, 28). The majority of SLE patients were recruited through the Hospital for Special Surgery (HSS) Autoimmune Disease Registry (29). The clinical characteristics and medical therapies of the lupus patients (such as treatments with glucocorticoids, prednisone, and hydroxychloroquine) have been previously described (11). Study subjects signed an informed consent approved by the Hospital for Special Surgery's Institutional Review Board that described laboratory investigation of patient material as well as review of concurrent and previous clinical and laboratory data. Some SLE patients were tested on multiple occasions.

Plasma samples. Twenty ml of heparinized blood was centrifuged and the plasma was removed and stored at −70° C. Clinical data were available on all SLE patients. Complete serologic data were available for fifty-nine patients (forty-three from the initial cohort and sixteen new SLE patients), and a composite score for titer of autoantibodies specific for RNA-binding proteins (anti-RBP, including anti-Ro, -La, -Sm, -RNP) was calculated.

WISH cell culture and stimulation. Human WISH epithelial cell line cells (American Type Culture Collection, #CCL-25, Manassas, Va.) were grown in Minimum Essential Medium supplemented with L-glutamine (2 mM), HEPES (20 mM), penicillin (100 μ/ml), streptomycin (100 μg/ml), and 10% fetal bovine serum (FBS) at 37° C., 5% CO₂. To measure IFIG-inducing activity in patient plasma, WISH cells were cultured at a density of 0.5×10⁵/0.1 ml in 96-well flat-bottomed plates with medium, recombinant human IFNα (rhIFNα; IFNαA, BioSource International, Camarillo, Calif.), rhIFNβ, -γ or -ω(R&D systems, Minneapolis, Minn.), or 50% donor plasma or serum, in the presence or absence of neutralizing antibodies to IFNα (polyclonal antibody [PBL Biomedical Laboratories]); monoclonal antibody (Chemicon International, Temecula, Calif.), IFNβ or IFNγ, or isotype control antibodies (R&D systems, Minneapolis, Minn.). After 6 hours or 20 hours of incubation, WISH cells were lysed and stored at −70° C.

Real-time PCR following reverse transcriptase polymerase chain reaction (RT-PCR). RNA was extracted from each cell lysate using the RNeasy Mini Kit (Qiagen, Valencia, Calif.). An aliquot of 0.4 μg of this RNA was reverse-transcribed to cDNA in a 20 μl reaction using SuperScript III RNase H-Reverse Transcriptase (Invitrogen, Carlsbad, Calif.). cDNA obtained from each sample was diluted 1:40, and 10 μl was amplified in a 25 μl real-time PCR reaction using 0.4 μM of sense and antisense primers and the 2X iQ SYBR Green Supermix (Bio-Rad, Hercules, Calif.). Hypoxanthine phosphoribosyltransferase 1 (HPRT1) was used as a housekeeping gene control. Primers for all target genes and HPRT1 were designed using Beacon Designer 2.06 software (Premier Biosoft International, Palo Alto, Calif.) in conjunction with the DNA mfold 3.1 program to exclude sequences with significant secondary structure (44).

SEQ ID NO:1 (IFIT1 forward),
5′-CTCCTTGGGTTCGTCTATAAATTG-3′;
SEQ ID NO:2 (IFIT1 reverse),
5′-AGTCAGCAGCCAGTCTCAG-3′;
SEQ ID NO:3 (IFI44 forward),
5′-CTCGGTGGTTAGCAATTATTCCTC-3′;
SEQ ID NO:4 (IFI44 reverse),
5′-AGCCCATAGCATTCGTCTCAG-3′;
SEQ ID NO:5 (PRKR forward),
5′-CTTCCATCTGACTCAGGTTT-3′;
SEQ ID NO:6 (PRKR reverse),
5′-TGCTTCTGACGGTATGTATTA-3′;
SEQ ID NO:7 (C1orf29 forward),
5′-AATCAGACAGAACAGTTAATCCTC-3′;
SEQ ID NO:8 (C1orf29 reverse),
5′-TCAACCATATCTTCAATGCTACC-3′;
SEQ ID NO:9 (MX1 forward),
5′-TACCAGGACTACGAGATTG-3′;
SEQ ID NO:10 (MX1 reverse),
5′-TGCCAGGAAGGTCTATTAG-3′;
SEQ ID NO:11 (MIG forward),
5′-CATCATCTTGCTGGTTCTG-3′;
SEQ ID NO:12 (MIG reverse),
5′-AGGATTGTAGGTGGATAGTC-3′;
SEQ ID NO:13 (HPRT1 forward),
5′-TTGGTCAGGCAGTATAATCC-3′;
SEQ ID NO:14 (HPRT1 reverse),
5′-GGGCATATCCTACAACAAAC-3′.

WISH cells cultured with medium were included as a control in each assay to provide a basis for normalization across experiments. Results for each experimental culture condition are expressed as relative expression (RE) compared to WISH cells cultured with medium.

Experiments may combine real-time, kinetic RT-PCR detection together with an internal homologous control that can be simultaneously detected alongside the target sequences. (See 47, for discussion of quantitative RT-PCR.). The advantages of real-time PCR include that it is quantitative, that only a small number of cells are required to generate sufficient cDNA for analysis, and the assay is simple to perform once the conditions for detecting a specific transcript are established.

The real-time quantitative amplification of gene expression and analysis is as described in Kirou et al. (10, 30) and is described generally as follows. Real-time PCR is performed using the Bio-Rad iCycle IQ Real-Time Detection System (Bio-Rad, Hercules, Calif.), although numerous other cyclers are available and suitable for use in the present methods. Quantitation is achieved by measuring the increase in fluorescence during the exponential phase of the amplification, which is in real time. An excitation system via an excitation wheel directs the light onto all of the wells of a 96-well plate. Next, fluorescent molecules from each well emit fluorescent light through an emission filter wheel and an image intensifier; this light is eventually detected by the CCD camera. The data are then transferred to a computer and analyzed with Bio-Rad software.

There are at least four methods used for real-time PCR: molecular beacons, hydrolysis probes (e.g. TaqMan), hybridization probes, and DNA-binding dyes (e.g. SYBR Green I), with any of these methods being suitable for steps in the present methods. The present results were obtained using the SYBR Green I method using the Bio-Rad iCycle IQ Real-Time Detection System with the target and housekeeping genes amplified in separate wells from the same template and by running a melt curve analysis of the PCR products at the end of the reaction.

cDNA standards are prepared for each gene to be amplified. Plasmids are typically used, but any known sample with a high expression for the desired gene can be used. For example, the cDNA standard for IFN-induced protein with tetraicopeptide repeats 1 (IFIT1) was made from a sample of 2 million PBMCs stimulated with 1000 U/mL of IFNα for 24 hours. Multiple aliquots of cDNA samples are made and frozen. Prior to PCR, cDNA dilutions for standard curves are made, typically by making three 10-fold dilutions in duplicate per plates. In later assays testing the performance of the reaction, typically only two dilutions are made. The 1:10 dilutions of all test/unknown cDNA samples are made and 10 μL (for more accurate pipetting) of each sample is added, in duplicate to wells in the 96-well PCR plate.

The Supermix for each primer is prepared as follows: (N+2)×12.5 μL of Bio-Rad SYBR Green Mix; (N+2) μL of the S and AS primer solution; and (N+2)×1.5 μL of DEPC-treated water. Next, 15 μL of the Supermix is added to each well. The final volume per reaction is 25 μL, with concentrations of 0.4 μM for each primer, of 25 U/mL of iTac DNA polymerase, of 0.2 mM each of dNTPs, and of 3 mM of Mg⁺⁺. The Supermix also contains SYBR Green I, a DNA intercalating dye responsible for the fluorescence emission during the reaction used to determine the starting quantity of the gene in question. As the cDNA is amplified during the successive reaction cycles, more and more dye binds to DNA, and more fluorescence is emitted. After the Supermix is added to each well, the plate is sealed with optical tape. Next, the plate is spun in a centrifuge at 300 g, at 4° C. for 3-5 minutes. The plate is covered with foil and kept refrigerated at 4° C. until it is placed into the machine. The time between preparation and run on the machine is minimized.

The PCR reaction is run according to manufacturer's instructions by placing the 96-well plate in the PCR machine and opening the iCycler software. The software is initiated by selecting the Library window and the View Protocol tab to select the most suitable protocol for the reaction. Typically, the cycle chosen is 2StepAmp+Melt.tmo, which includes five cycles. Cycle 1 consists of 1 repeat and 1 step with dwell time of 3 minutes (min.) at 95° C. Cycle 2 consists of 40 repeats of step 1 (10 second (sec) dwell time at 95° C.) and step 2 (45 sec of dwell time at 55° C.). Cycles 3 and 4 each have 1 repeat and 1 step, with 1 min. dwell times at 95° C. and 55° C., respectively. Finally, cycle 5 consists of 80 repeats of step 1 with a 10 sec dwell time starting at 55° C. and positive increments of 0.5° C. for every repeat. By clicking on Edit This Protocol, the settings of the various cycles may be changed according to the experimental conditions. The methods used for analyzing the IFIGs of the present conditions used the above cycles with the annealing temperature of 58° C.

Two genes are amplified: a target gene and a housekeeping gene, which serves as the internal control for each sample. HPRT1 is the housekeeping gene used in the present experiments (previously described and available at GenBank Accession No. NM_{—}000194, SEQ ID NO:26 with deduced amino acid sequence SEQ ID NO:27). The specificity of the PCR reactions for the amplified genes is confirmed by examining the melting curve (generated during cycle 5 of the reaction) for each gene amplification.

The data is analyzed using Bio-Rad iCycle iQ software (version 3.0a). Optionally, this analysis can be done on a personal computer away from the PCR machine using the appropriate software. The View Post Run Data tab is selected, the experiment file is selected, and then Analyze Data is selected to obtain results. The amplication curves from all wells are displayed on the Data Analysis window under the PCR Quantification tab (the x-axis represents the PCR cycle and the y-axis represents the fluorescence intensity). The melting curves and the standard curves can also be viewed by clicking on the corresponding tabs.

To assess the expression of each gene for all unknown samples, the corresponding wells for that gene (unknowns and standards) are selected, the Select Wells box is selected (highlighting the relevant wells), and Analyze Selected Wells is clicked. The program then automatically sets the best possible threshold line that will horizontally intersect all amplification curves at their exponential phase. This can be best visualized when Analysis Mode is set at the PCR Baseline Cycle Subtracted option and with the graph adjusted so that the fluorescence intensity axis is expressed in a Log scale. The threshold line will determine the threshold cycle of amplification for each sample and will plot it (y-axis) against the Log Starting Quantity of the particular gene x-axis). The latter value is known for the standards (and typically is set a 1, 10, or 100 according to their dilutions) and is calculated for the unknowns in the standard curve view of the results. The standard curve also gives the correlation coefficient (optimally above 0.98) and the PCR efficiency (optimally between 80 and 120%).

The curves typically show only one melting peak for each amplified gene. If more than one peak is present, a primer dimer (melt peak lower than amplicon peak) may have formed, or another gene may have been concurrently amplified. Either of these situations is undesirable and the results would be discarded.

The threshold cycle values for the target and reference genes for each sample are entered into an Excel file and subtracted from the corresponding values of a reference sample (usually the one with no stimulation). These differences are then used as exponents with base the sum of the efficiency of the PCR reaction expressed as a decimal plus 1 (i.e., for efficiency of 98%, this would be 1.98). By definition, therefore, the corresponding differences of the reference sample for both the target and housekeeping genes will be 0, which when used as exponents will result in values of 1. Finally, the target gene values are divided by the housekeeping gene values for each sample, and the result is the relative expression value for each unknown sample.

In an example of determining IFIT1 target gene expression, PBMCs from a healthy donor were either left untreated or were cultured with 200 U/mL IFNα Two of the IFNα treated samples were also treated with anti-IFNα Ab at a concentration of 200 neutralization units/mL or an isotype control Ab. Cell lysis, RNA isolation, reverse transcription, and real-time PCR amplification followed for IFT1 and HPRT1 (the housekeeping gene). The amplification, melt, and standard curves for each gene were determined as described above. The efficiency for each reaction was determined as 111.4% or E=1.114 for IFIT1 and 96.8% or E=0.968 for HPRT1. The threshold cycles (Ct) for each sample and for each gene were calculated. The ΔCt or differences of the Ct of each cycle, from the Ct of medium (reference sample) for the two genes were determined. The E+1 for each gene (2.114 and 1.968) was also calculated as raised to the corresponding ΔCt power for each sample (E+1)^{ΔCt}. The relative expression (RE) of IFIT1 for the sample was determined by dividing its (E+1)^{ΔCt} value by the (E+1)^{ΔCt} value of HPRT1.

The mean (M) and SD of each IFIG for the group of healthy donors (M_{HD} and SD_{HD}) were used to calculate that gene's expression score for each study subject, defined as the number of SD_{HD} above the M_{HD}. Cumulative IFNα scores, representing the sum of scores for each of the tested genes preferentially induced by IFNα and for genes preferentially induced by IFN-γ were derived from each subject. An IFNα score was considered high if it fulfilled one of the two following criteria: 1) expression of at least 2 of the 3 IFNα genes at a level ≧2 SD_{HD} above the M_{HD}; 2) expression of a single IFNα gene at a level ≧4 SD_{HD} above the M_{HD}. The IFNγ score was considered high or low based on analogous criteria for the desired IFNγ genes.

Statistical analysis. Two-group comparisons of continuous data that had a normal distribution were assessed using t-tests. The Kruskal-Wallis nonparametric analysis was used to compare the 3 study groups for the values of the 5 interferon-inducible genes (IFIG), because the data were not normally distributed. Correlation and linear regression analysis were performed to detect relationships between WISH cell data and IFN score or anti-RBP and anti-dsDNA autoantibody titers.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Induction of IFIG in an in vitro WISH cell system by recombinant human interferon: time course and dose response. To develop an in vitro assay for quantification of IFIG-inducing activity in patient plasma or serum, WISH cell line cells, previously demonstrated to be IFN-responsive (31), were cultured with rhIFNα, rhIFNγ or SLE plasma. Expression of five genes, interferon-induced protein with tetratricopeptide repeats 1 (IFIT1, SEQ ID NO:15,), interferon-induced protein 44 (IFI44, SEQ ID NO:16, deduced amino acid sequence SEQ ID NO:17), protein kinase, interferon-inducible double stranded RNA dependent (PRKR, SEQ ID NO:18, deduced amino acid sequence SEQ ID NO:19) also known as eukaryotic translation initiation factor 2-alpha kinase 2 (EIF2AK2)), C1orf29 (SEQ ID NO:20, deduced amino acid sequence SEQ ID NO:21) and myxovirus (influenza virus) resistance 1 (MX1, SEQ ID NO:22, deduced amino acid sequence SEQ ID NO:23) was measured in cultured WISH cells. These five genes were previously determined to be preferentially induced by IFNα in patient PBMC cells. The sequences of these five genes have been described and are available from the GenBank Database as follows: SEQ ID NO: 15 is found at Accession No. NM_{—}001548; SEQ ID NO: 16 is found at Accession No. NM_{—}006417; SEQ ID NO:18 is found at Accession No. NM_{—}002759; SEQ ID NO:20 is found at Accession No. NM_{—}006820; and SEQ ID NO:22 is found at Accession No. NM_{—}002462.

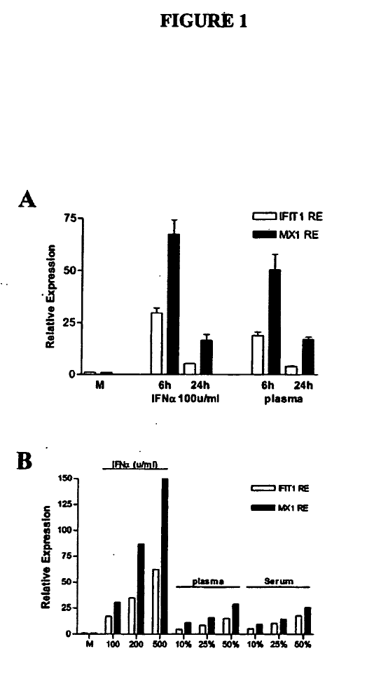

IFIT1, IFI44, PRKR, C1orf29 and MX1 mRNAs were preferentially induced by IFNα, with MX1 most sensitive to stimulation with rhIFNα (Table 1). Both IFIT1 and MX1 showed rapid expression after 6 hours of WISH cell culture with rhIFNα (FIG. 1A), and rhIFNω and rhIFNβ (other type I IFNs) induced these IFIG with similar kinetics (data not shown). IFIT1 and MX1 were induced in WISH cells by rhIFNα in a dose related manner (FIG. 1B). One IFIG, CXCL9 (also known as monokine induced by gamma interferon (MIG)), was strongly induced in WISH cells by rhIFNγ and not at all induced by rhIFNα after a 20 hr incubation period (Table 1). The sequence for MIG (SEQ ID NO: 24, deduced amino acid sequence SEQ ID NO:25) has been described and is available at the GenBank Database Accession No. NM_{—}002416.

TABLE 1
The genes preferentially induced (e.g. exhibiting increased expression
when compared to control cells not exposed or contacted
with IFNα) in WISH cells by IFNα were
IFIT1, IFI44, PKR, C1orf29, MX1. The genes preferentially
induced (e.g. exhibiting increased expression when compared
to control cells not exposed or contacted with IFNγ)
in WISH cells by IFNγ included MIG. 500 μ/ml of
rhIFNα or rhIFNγ were incubated with WISH cells
for 20 hrs and expression of IFIG was determined by quantitative
real-time PCR as described above. Relative expression of each
gene is shown. Data are from one of three similar experiments.

| | IFIT1 | IFI44 | PRKR | C1orf29 | MX1 | MIG |
|---|---|---|---|---|---|---|
| | RE | RE | RE | RE | RE | RE |
| IFNα500 u/ml | 12.79 | 3.01 | 4.49 | 28.92 | 138.2 | 0.59 |
| IFNγ500 u/ml | 2.79 | 2.87 | 1.37 | 11.14 | 23.03 | 995.73 |

WISH cells were cultured in vitro with SLE patient plasma and tested for IFIG expression. SLE plasma or serum used at 50% volume/volume concentration induced IFIT1 and MX1 with a similar kinetics to that seen with rhIFNα and with activity at a level similar to that of 100 u/ml rhIFNα (FIGS. 1A and 1B). 100 u/ml rhIFNα or SLE plasma at 50% volume/volume concentration was incubated with WISH cells for either 6 hours (hrs) or 24 hrs and expression of IFIG determined by quantitative real-time PCR as described above. Relative expression of two IFNα-inducible genes (IFIT1 and MX1) is shown as mean±SEM of data from three similar experiments. The plasma data represent the same sample tested on 3 distinct occasions. Three different concentrations of rhIFNα (100 u/ml, 200 u/ml, 500 u/ml) or 3 concentrations (10%, 25%, 50%) of plasma or serum from a lupus patient were incubated with WISH cells for 6 hrs and IFIG expression assayed. Relative expression of two IFNα-inducible genes (IFIT1 and MX1) is shown as data from one of two similar experiments.

Lupus plasma and serum induced IFIG expression in the WISH cells to a similar degree (FIG. 1B). The IFIG-inducing activity of rhIFNα and that present in SLE sera were heat labile, with activity reduced by 62-99% after incubation at 56° C. for 30 minutes. The results demonstrate that WISH epithelial cell line cells, or other similar cell lines capable of IFIG induction, along with quantitative real-time PCR, can be used to assay components of patient plasma or serum that mediate activation of the IFN pathway.

### Example 2

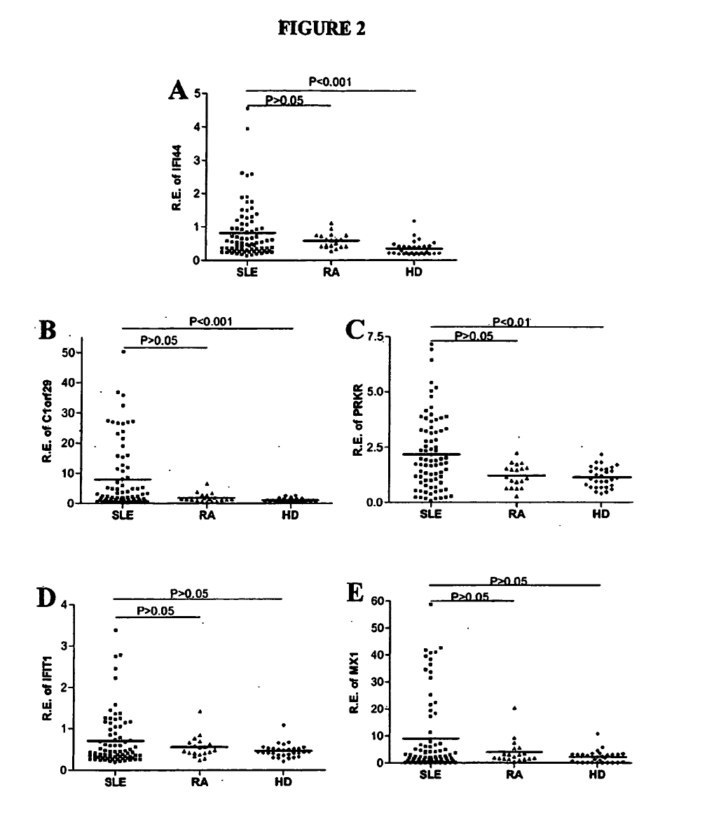

Correlation of lupus plasma activity with expression of IFIG in SLE PBMC. To assess the level of IFIG-inducing activity in plasmas from a diverse population of SLE patients, as well as in plasmas from disease controls with RA and from healthy subjects, WISH cells were cultured with medium or with plasma from seventy-three SLE patients, nineteen RA patients and thirty healthy donors and IFIG expression was measured by real-time PCR. Five IFNα-inducible genes and one IFNγ-inducible gene were quantified. Plasma samples from patients with SLE (n=73) or RA (n=19), or from healthy donors (n=30), were incubated with WISH cells at a 50% volume/volume concentration for 20 hrs. WISH cells were then lysed and used for RNA isolation, reverse transcription, and amplification by quantitative real-time PCR. Relative expression of five IFNα-induced genes is shown. Mean values for each group are indicated by the horizontal line shown in FIG. 2. P values for differences between SLE patients and either healthy controls or RA patients are indicated.

As seen in FIG. 2, about one third of SLE patients demonstrated high plasma IFIG-inducing activity for all 5 IFNα-inducible genes tested. When expression of one IFIG was plotted against expression of each of the other four IFIGs in WISH cells cultured with SLE plasma, a significant correlation was demonstrated in each case (r values ranging from 0.36 to 0.89) as shown in FIG. 2. When the three study groups were compared, the induction of IFI44, C1orf29, and PRKR genes was significantly higher in SLE patients compared to healthy donors (FIG. 2). The mean relative expression of MIG induced by SLE plasmas was <1, indicating minimal or no functional IFNγ activity in those samples.

### Example 3

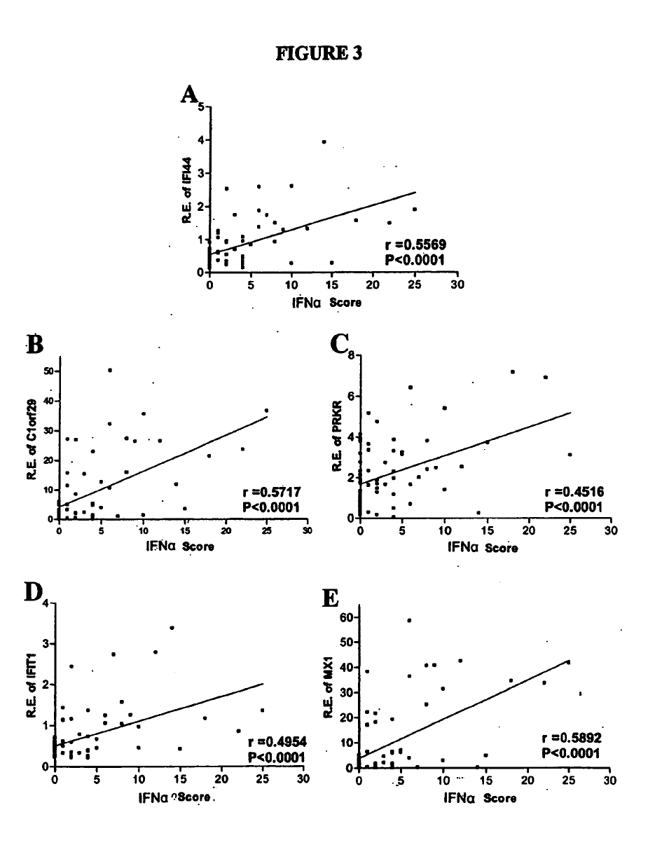

Correlation of IFNα-inducible genes in WISH cells cultured with SLE plasma with the level of mRNA encoded by IFNα-inducible genes in PBMC cells from patients. The expression of each of these five IFNα-inducible genes in WISH cells cultured with SLE plasma was correlated with the level of mRNA encoded by IFNα-inducible genes in the PBMC of those same patients collected on the same day, as expressed as an IFNα score (FIG. 3). Relative expression of 5 IFNα-induced genes in WISH cells cultured with 50% lupus plasma was plotted against the IFNα score, previously assayed based on real-time PCR analysis of patient PBMC cells. The IFNαscore was calculated based upon previous real-time PCR analysis of expression of three IFNα-inducible genes (IFIT1, IFI44, PRKR) in patient PBMC. The expression of IFIT1, IF44, and PRKR in patient PBMC was described in detail by Kirou et al. 2005 (11). The Spearman rho(r) and p correlation values are shown. The p value for each of the five IFNα-inducible genes was <0.0001, suggesting that the IFIG expression detected in SLE PBMC is likely to be attributed to stimuli present in patient plasma.

Inhibition of SLE plasma activity with anti-IFNα antibodies. To identify the components in SLE plasma responsible for IFIG expression in WISH cells, a rabbit polyclonal anti-IFNα antibody, as well as monoclonal anti-IFNα, -βand -γ antibodies, were tested for inhibitory activity against the relevant recombinant IFNs. Polyclonal anti-IFNα antibody effectively inhibited the induction of IFIT1 and MX1 by rhIFNα, as did monoclonal anti-IFNα antibody. Monoclonal anti-IFNβ and -γ antibodies were also specific for IFN-β and -γ, respectively, and were only minimally cross-reactive with the other IFNs.

### Example 4

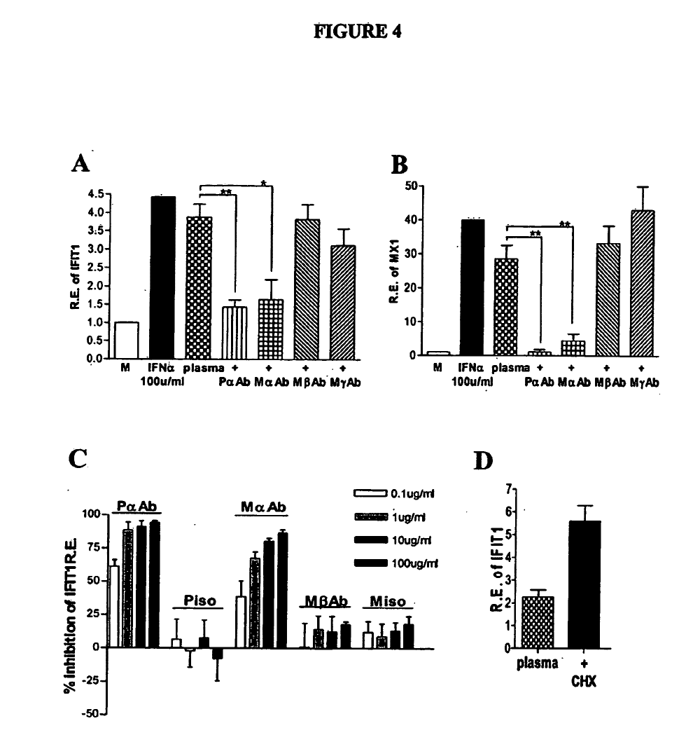

Ability of antibodies to inhibit IFN activity in SLE plasmas. The capacity of the antibodies described in Example 3 to inhibit the activity in SLE plasmas was assessed. Relative expression of two IFNα-inducible genes (IFIT1, MX1) is shown in FIGS. 4A and 4B, respectively. Both polyclonal and monoclonal anti-IFNα antibodies significantly inhibited IFIT1 expression induced by SLE plasma (FIG. 4A). The polyclonal and monoclonal anti-IFNα antibodies nearly ablated MX1 expression (FIG. 4B). For the data shown in FIGS. 4A and 4B, WISH cells were incubated for 6 hrs with rhIFNα 100 u/ml or with 50% lupus plasma together with medium, rabbit polyclonal anti-IFNαAb (PαAb), 2.5 μg/ml, or with monoclonal anti-IFNαAb(MαAb), 29.6 μg/ml, anti-IFNβAb (MβAb), 10 μg/ml, or anti-IFNγAb (MγAb), 3 μg/ml, and IFIG expression was determined. All antibody doses could maximally inhibit the induction of IFIG by 500 u/ml of its corresponding rhIFN and at the same time showed only minimal cross-reactivity with the other IFNs. Results for FIGS. 4A and 4B are the mean±SEM represented by 3 different lupus patients; *p<0.05 and **p<0.01.

Dose response studies showed that 1 μg/ml of monoclonal anti-IFNα antibody resulted in more than 50% inhibition of SLE plasma-induced IFIG expression (FIG. 4C). In contrast to the results with anti-IFNα antibodies, anti-IFNβ and -γantibodies had minimal effects on WISH cell IFIG expression induced by SLE plasma (FIGS. 4A, 4B and 4C). For the results shown in FIG. 4C, WISH cells were cultured with 50% lupus plasma in the presence of varying concentrations of polyclonal anti-IFNα antibody (PαAb), control rabbit IgG (Piso), monoclonal anti-IFNα antibody (MαAb), monoclonal anti-IFNβ antibody (MβAb), or mouse isotype control IgG1 (Miso) and relative expression of IFIT1 was determined. Results are expressed as % inhibition compared to expression induced by 50% SLE plasma. Results are the mean±SEM of 3 lupus patients. In addition, preliminary experiments using a monoclonal antibody confirmed to be specific for IFNλ1 (IL-29), a member of the type III IFN family, showed no inhibition of IFIG-inducing activity in SLE plasma. It was possible that inducers of IFNα, rather than IFNα itself, were indirectly responsible for WISH cell IFIG expression.

To determine whether production of new protein was required for expression of IFIG in WISH cells cultured with SLE plasma, WISH cells were pre-cultured in cycloheximide to inhibit protein synthesis, followed by culture with SLE plasma for 24 hrs. Cycloheximide did not reduce the capacity of SLE plasma to stimulate IFIG expression in WISH cells, and in fact increased IFIG expression, suggesting that IFNα protein itself, rather than a stimulus for IFNα production, directly accounted for activation of IFIG in WISH cells by lupus plasma (FIG. 4D). For the data shown in FIG. 4D, WISH cells were pre-incubated with cycloheximide, 10 μg/ml, prior to addition of SLE plasma. After 24 hrs of culture, IFIT1 expression was determined. Mean±SEM for 5 experiments is shown.

### Example 5

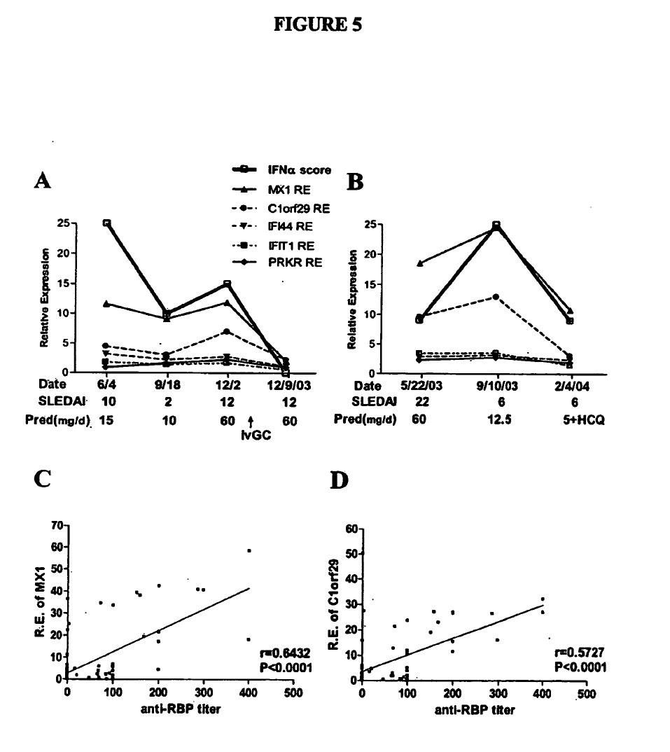

Longitudinal study of IFNα activity in SLE plasma. To determine whether IFN activity in SLE plasma varies over time and parallels IFNα score, reflecting IFNα-inducible gene expression in patient PBMCs, sequential plasma samples were collected from two SLE patients over 5 or 7 months. The relative expression of IFNα-inducible genes induced in WISH cells by patient plasma varied over time and was associated with a similar pattern of IFN-inducible gene expression in the patients' PBMC, as reflected in the IFNα score (FIGS. 5A and 5B).

For the data shown in FIG. 5A, plasma samples from lupus patient #749 were collected on Jun. 4, 2003, Sep. 18, 2003, Dec. 2, 2003, and Dec. 9, 2003. For the data shown in FIG. 5B, plasma samples from lupus patient #775 were collected on May 22, 2003, Sep. 10, 2003 and Feb. 4, 2004. For both patients' samples, IFN activity in plasma was quantified in the WISH cell assay and relative expression of 5 IFNα-inducible genes was plotted over time, along with the IFNα score determined by analysis of patient PBMC samples collected on the same dates. SLEDAI score and changes in medical therapy at each time point are indicated. For patient #749, intravenous pulse glucocorticoid therapy (ivGC) was given as 1 gram of intravenous methylprednisolone 4 days prior to the last time point (Dec. 9, 2003). For the data shown in FIG. 5C, and FIG. 5D, relative expression of 2 IFNα-inducible genes (FIG. 5C, MX1 and FIG. 5D, C1orf29) in WISH cells induced by 50% lupus plasma is plotted against anti-RBP autoantibody titer in those same patients. R and p values are shown.

Of the five IFIG measured, MX1 most closely tracked the IFNα score. Documentation of SLEDAI score suggested a relationship between IFIG-inducing plasma activity and IFNα score and disease activity in the patient data shown in FIG. 5A. Administration of intravenous pulse glucocorticoids ablated both measures of IFN pathway activity in that patient. Fluctuations in the dose of prednisone or addition of hydroxychloroquine may have complicated interpretation of a relationship between plasma IFN activity and systemic lupus erythematosus disease activity index (SLEDAI) score in the patient data shown in FIG. 5B.

### Example 6

Correlation of IFIG expression with anti-RNA-binding protein specific autoantibody titer. It had previously been observed that elevated IFIG expression in SLE PBMC was highly associated with the presence of autoantibodies specific for RNA-binding proteins (11). In the present study, a strong correlation was found between the level of expression of MX1 and C1orf29 mRNA in WISH cells cultured with SLE plasma and the aggregate anti-RBP autoantibody titer (titer of anti-Ro plus anti-La plus anti-Sm plus anti-RNP) in the serum of SLE patients (FIGS. 5C and 5D). To strengthen support for a relationship between IFN activity and production of anti-RBP autoantibodies, WISH assay data were separately analyzed in those patients (n=43) previously studied and reported to show a significant association between a high IFNα score and presence of anti-RBP autoantibodies (11). In that initial cohort, a strong correlation was observed between MX1 expression in the WISH assay and anti-RBP titer (r=0.6468; p<0.001). To provide confirmation of that relationship in a second patient group, data from 16 patients not previously studied were analyzed. The correlation between MX1 mRNA expression induced by plasma in the WISH cell assay with anti-RBP titer in that second patient cohort was equally significant (r=0.6595; p<0.001).

Previous studies of IFIG expression in PBMC showed a negative correlation between IFNα score and serum C3 level. The present data are consistent with this observation; expression of MX1 in WISH cells cultured with SLE plasma showed a negative correlation with serum C3 (r=−0.3564; p=0.003).

IFNα-regulated genes were induced by SLE plasmas but not by most RA or healthy control plasmas. The activity in SLE plasma was inhibited >90% by anti-IFNα antibody, but not by anti-IFN or anti-IFNγ antibodies. The expression level of each IFNα target gene induced by SLE plasma correlated with expression of those genes in PBMC of the same patients studied ex vivo and with anti-RBP specific autoantibody titer. Plasma activity paralleled PBMC expression of IFNα-inducible genes over time.

IFNα in SLE plasma is a major stimulus for IFN target gene expression and correlates to expression of those genes in lupus PBMC and to titer of anti-RBP autoantibodies. These data provide additional support for IFNα mediating immune system activation and dysregulation in SLE.

The assays described herein, using WISH cells as sensitive targets of type I IFNs in body fluids, are useful for quantification of the activity of those IFNs in patients and their relationship to serologic and clinical features of disease. These cellular assays are superior to ELISA assays which have proved either insensitive or unreliable. Tests such as ELISAs cannot detect the full complement of IFIG-activating IFNs or provide significant correlative data with clinical or serologic features of disease.

In one example, the methods provide a functional assay of IFN activity showing that IFNα is the IFN isoform responsible for the majority of IFIG expression in SLE and that SLE plasma IFN activity is highly correlated with anti-RBP autoantibody titer.

The present methods provide new functional assays that allow characterization of the plasma components responsible for IFN pathway activation in SLE. These assays are based on the capacity of plasma or serum from a subject to induce IFIG expression in responsive target cells, that are not the subject's cells. Previous studies described using WISH or other cell lines to measure the effect of IFN on cell viability in the setting of virus infection (37). While sensitive, these viral assays do not allow direct comparison of IFN activity in plasma with activation of IFIG, nor do they permit analysis of other components of plasma, in addition to IFN, that might impact IFIG expression. The assays described herein, using quantitative real-time PCR analysis of human WISH epithelial cell line cells cultured with patient plasma, are shown to be sensitive and reliable for detection of IFN activity in SLE plasma and serum. The WISH cell line expresses high levels of IFIG upon in vitro stimulation with IFNα, -β, and -γ and the level of this expression is dose related. The WISH cell line shows similar responses to rhIFNα and to SLE plasma, supporting its relevance for quantification of IFN in SLE patient samples.

Although the role for IFNα in lupus has been reported for many years and has been supported by abundant data, most of those studies have only investigated IFNα, considered the prototype type I IFN. In fact, the gene targets of the various IFNαisoforms, as well as those of IFNβ and IFNω, are largely similar. IFNα has been less well studied than IFNα, but early reports suggest that it activates similar genes to IFNαand -β, (16,17). Type II IFN, or IFNγ, may be expressed locally at sites of tissue inflammation, as in lupus nephritis, but high levels of circulating IFNγ have not been reproducibly observed. Previous studies in which IFIG expression was detected in SLE patient PBMC cells, indicated that few lupus patients show evidence of increased expression of IFIG that are preferentially induced by IFNγ (10,11).

The present results indicate a role for IFNα in SLE plasma, since both polyclonal and monoclonal anti-IFNα antibodies inhibited much of the IFIG inducing activity in SLE plasma. Monoclonal antibodies confirmed to be specific for IFNβ did not inhibit the lupus plasma activity, and preliminary data suggest that IFNα does not contribute to the activation of WISH cells by SLE plasma. The role of IFNωcould not be adequately tested as the commercially available antibody proved nonspecific. The expression of MIG, a gene highly sensitive to induction by IFNγ, was not increased in WISH cells cultured with lupus plasmas, suggesting that if present at all, IFNγ levels were not sufficient to activate that very responsive gene. Further supporting a role for IFNα is the observation that plasma activity across the entire tested SLE patient population correlated with expression of IFNα-inducible genes in PBMC of those patients, as measured by an IFNα score. In addition, plasma IFIG-inducing activity and PBMC IFNα score showed parallel patterns when measured in plasma and PBMC samples collected longitudinally and showed potential for demonstrating an association with quantitative measures of disease activity. Thus, although there may be small amounts of other IFNs present in SLE patient plasma, the present results indicate that IFNα, rather than IFNβ or IFN γ, is the predominant IFN type. There could be conditions under which another type I IFN, such as IFNωor IFNγ are present, or when one set of IFNα subtypes predominates over another set of IFNα subtypes. Specific antibodies or specific inhibitors can be used in conjunction with the present assay system to identify and confirm the major IFN types in any body fluid sample.

IFNα appeared to be directly responsible for IFIG expression as the protein synthesis inhibitor, cycloheximide, did not abrogate the activity in lupus plasma. Together, these data demonstrate that the WISH assay system can be used to quantify and characterize mediators such as IFN isoforms present in patient plasma.

In earlier studies of SLE patient PBMC cells, logistic regression analysis of clinical and serologic data showed that the presence of autoantibodies specific for RNA-binding proteins (RBP: Ro, La, Sm or U1RNP) was independently associated with high expression of IFNα-inducible genes in lupus PBMC. Consistent with the proposed functional link between plasma activity and IFNα-inducible gene expression in SLE PBMC, IFIG expression in WISH cells cultured with SLE plasma was highly correlated with the titer of anti-RBP autoantibodies (the sum of the titers of the four component specificities). Among them, anti-Ro antibody showed the strongest correlation. This observation was further supported when WISH assay data were analyzed separately in two patient cohorts, those previously characterized in studies of IFIG expression in PBMC and a second cohort of newly recruited SLE patients. In both groups, MX1 expression induced by patient plasma was significantly correlated with anti-RNP autoantibody titer. In contrast to anti-RBP, anti-dsDNA autoantibody titer showed no significant correlation with IFIG expression assayed in WISH cells induced by SLE plasma.

### Example 7

Type I Interferon Expression in Ro/La Autoantibody Positive Mothers. In vitro experiments have shown that sera containing anti-RNA binding protein (anti-RBP) antibodies reactive with Ro and La are capable of inducing type I IFN production in dendritic cells when combined with dead cellular material. As described above, in many SLE patients anti-RBP antibodies are associated with increased type I IFN. Women with Ro/La autoantibodies are at risk of having a child with neonatal SLE, and these women often have signs and symptoms of connective tissue disease themselves.

The association of Ro/La autoantibodies and type I IFN activating capability of patient serum in pregnant women was studied. (43) Serum samples from 66 mothers with Ro/La autoantibodies were obtained from the Research Registry for Neonatal Lupus. The mothers were recruited to the registry if they were known to have Ro/La autoantibodies and became pregnant, or if they gave birth to a child with neonatal SLE. Type I IFN is detected using an interferon reporter cell assay of the present invention. Cells are exposed to serum for 6 hours, cDNA is made from total cellular mRNA, and the relative expression of three genes induced by type I IFN signaling are quantified by real time PCR. The relative expression values are compared with healthy donor sera in the same assay. Type I IFN data are correlated with clinical data for each patient. The 66 mothers had varying diagnoses including SLE, Sjogren's Syndrome (SS), pauci-SLE, pauci-SS, SLE/SS, and some were asymptomatic. The prefix “pauci-” indicates that the patient meets some disease criteria, but not enough to be formally classified. All had Ro and/or La autoantibodies.

The results from these studies showed high type I IFN expression in 8 of 22 patients (36%) with established SLE or SLE/SS and 5 of 13 (38%) with SS. Only 1 of 16 individuals (6%) that were asymptomatic had high type I IFN, despite high titers of Ro and/or La autoantibodies. Six of 8 patients (75%) with pauci-SLE had increased type I IFN expression, while none of the 7 patients with pauci-SS had increased type I IFN expression. All of the pauci-SLE patients were photosensitive, while only 50% of the SLE and SLE/SS patients were photosensitive. Three of the pauci-SLE mothers developed SLE at a mean follow up of 56.9 months. Four of the 16 asymptomatic patients progressed to either SLE (2) or SS (2), however high type I IFN was not detected in the asymptomatic phase in these 4 patients.

These studies showed that elevated type I IFN activity was found in the majority of serum from SLE and SS patients, but was very uncommon in asymptomatic patients, despite high titer Ro and/or La autoantibodies. This suggests that although sera containing Ro and La autoantibodies can help trigger type I IFN production in vitro, additional factors related to the disease processes in SLE and SS are important in type I IFN production in vivo. High type I IFN expression was seen in the highest frequency in the pauci-SLE group, and this group also had a high rate of photosensitivity. High type I IFN was not seen in the pauci-SS group. Thus, reporter cell assays of the present invention will be useful in detecting elevated type I IFN activity in the serum from pregnant SLE and SS patients and for monitoring disease manifestation in pregnant patients with SS and SLE.

### Example 8

Molecular Pathways Associated with Progression of Carotid Atherosclerosis in SLE. Premature development of atherosclerosis in patients with systemic lupus erythematosus (SLE) is a disease manifestation thought to be due to chronic systemic inflammation, but the molecular pathways that account for this complication have not yet been determined. Patients with rapid progression of carotid plaque were studied to identify SLE-associated mediators of accelerated atherosclerosis (45).

For these studies, SLE patients enrolled in a longitudinal study of pre-clinical atherosclerosis underwent serial carotid ultrasound and clinical assessment at two time points separated by an average of 33+9 months. The 38 patients who demonstrated carotid plaque on the second study were separated into 2 groups: progressors, defined as patients with new plaque lesions compared to their first visit (n=21), and non-progressors, defined as patients with stable plaque compared to their first visit (n=17). Expression of genes representing the interferon (IFN) and inflammatory pathways was quantified in PBMC by real-time PCR.

Results of these studies showed that progressors and non-progressors were comparable with regard to age, sex, race, and number of plaques on the second visit. On bivariate analysis, progressors expressed IFN-inducible genes (C1ORF29, IFIT1, IFI44, IRF7, guanylate nucleotide binding protein 1(GBP1)) and several inflammatory genes (IL-8, PBEF) at higher levels than non-progressors. Levels of lipids and presence of other cardiovascular risk factors (hypertension (HTN), diabetes mellitus (DM), smoking, and body mass index (BMI)) were similar in progressors and non-progressors. Logistic regression multivariate analysis confirmed the independent positive association of an IFN-inducible gene, C1ORF29 (p=0.058), and IL-8 (p=0.066) with plaque progression.

Rapid progression of atherosclerosis in SLE is associated with activation of the IFN pathway, as well as other proinflammatory pathways culminating in inflammatory cytokine induction of IL-8, and is independent of traditional cardiovascular risk factors. These data extend the clinical relevance of the IFN pathway in SLE to the cardiovascular system and suggest that therapeutic interventions targeting these pathways may arrest the progression and morbidity/mortality associated with atherosclerosis in SLE. Reporter cell assays of the present invention will be useful alone or in combination with other clinical observations, for determining particular manifestations of disease, such as the rapid progression of atherosclerosis in SLE patients as being indicative of being a progressor or a non-progressor.

### Example 9

Expression of IFNα Inducible Genes in Primary Sjogren's Syndrome. Since IFNα may contribute to the progression and development of lymphoproliferative disease in Sjogrens's patients, the interferon assays of the present invention are expected to be useful for diagnosing, monitoring, and determining efficacy of test compounds for individuals with Sjogrens's syndrome. Patients with primary Sjogrens's syndrome (pSS) can be differentiated into those who develop a lymphoproliferative disorder that can lead to significant mortality, and those with a benign prognosis. It has been shown that numerous type I interferon genes related to virus infection were found among the top 200 genes with increased expression in primary SS (46). Thus, this gene expression profile from a body fluid sample from a patient suspected of having pSS would indicate that the patient would be at risk for developing lymphoma. The methods of the present invention utilizing a reporter cell system as an interferon assay would be especially useful for determining which SS patients are likely to develop a lymphoproliferative disorder leading to lymphoma. Additionally, the methods of the present invention will be useful to diagnose, monitor, and determine the efficacy of test compounds for individuals with Sjogrens's syndrome.

### REFERENCES

1. Hooks J J, Moutsopoulos H M, Geis S A, Stahl N I, Decker J L, Notkins A L. Immune interferon in the circulation of patients with autoimmune disease. N Engl J Med 1979; 301(1):5-8.
2. Preble O T, Black R J, Friedman R M, Klippel J H, Vilcek J. Systemic lupus erythematosus: presence in human serum of an unusual acid-labile leukocyte interferon. Science 1982; 216(4544):429-31.
3. Ronnblom L E, Alm G V, Oberg K E. Possible induction of systemic lupus erythematosus by interferon-alpha treatment in a patient with a malignant carcinoid tumour. J Intern Med 1990; 227(3):207-10.
4. Gota C, Calabrese L. Induction of clinical autoimmune disease by therapeutic interferon-alpha. Autoimmunity 2003; 36:511-8.
5. Blanco P, Palucka A K, Gill M, Pascual V, Banchereau J. Induction of dendritic cell differentiation by IFN-alpha in systemic lupus erythematosus. Science 2001; 294(5546): 1540-3.
6. Baechler E C, Batliwalla F M, Karypis G, Gaffney P M, Ortmann W A, Espe K J, et al. Interferon-inducible gene expression signature in peripheral blood cells of patients with severe lupus. Proc Natl Acad Sci USA 2003; 100(5):2610-5.
7. Bennett L, Palucka A K, Arce E, Cantrell V, Borvak J, Banchereau J, et al. Interferon and granulopoiesis signatures in systemic lupus erythematosus blood. J Exp Med 2003; 197(6):711-23.
8. Han G M, Chen S L, Shen N, Ye S, Bao C D, Gu Y Y. Analysis of gene expression profiles in human systemic lupus erythematosus using oligonucleotide microarray. Genes Immun 2003; 4(3):177-86.
9. Ye S, Pang H, Gu Y Y, Hua J, Chen X G, Bao C D, et al. Protein interaction for an interferon-inducible systemic lupus associated gene, IFIT1. Rheumatology (Oxford) 2003; 42(10):1155-63.
10. Kirou K A, Lee C, George S, Louca K, Papagiannis I G, Peterson M G, et al. Coordinate overexpression of interferon-alpha-induced genes in systemic lupus erythematosus. Arthritis Rheum 2004; 50(12):3958-67.
11. Kirou K A, Lee C, George S, Louca K, Peterson M G, Crow M K. Activation of the interferon-alpha pathway identifies a subgroup of systemic lupus erythematosus patients with distinct serologic features and active disease. Arthritis Rheum 2005; 52:1491-503.
12. Crow M K, Kirou K A, Wohlgemuth J. Microarray analysis of interferon-regulated genes in SLE. Autoimmunity 2003; 36(8):481-90.
13. Crow M K, Wohlgemuth J. Microarray analysis of gene expression in lupus. Arthritis Res Ther 2003; 5(6):279-87.
14. Dall'era M C, Cardarelli P M, Preston B T, Witte A, Davis J C. Type I interferon correlates with clinical and serologic manifestations of systemic lupus erythematosus. Ann Rheum Dis. Published online Apr. 20, 2005.
15. Hardy M P, Owczarek C M, Jermiin L S, Ejdeback M, Hertzog P J. Characterization of the type I interferon locus and identification of novel genes. Genomics 2004; 84:331-45.
16. Kotenko S V, Gallagher G, Baurin V V, Lewis-Antes A, Shen M, Shah N K, et al. IFN-lambdas mediate antiviral protection through a distinct class II cytokine receptor complex. Nat Immunol 2003; 4:69-77.
17. Sheppard P, Kindsvogel W, Xu W, Henderson K, Schlutsmeyer S, Whitmore T E, et al. IL-28, IL-29 and their class II cytokine receptor IL-28R. Nat Immunol 2003; 4:63-8.
18. Vallin H, Perers A, Alm G V, Ronnblom L. Anti-double-stranded DNA antibodies and immunostimulatory plasmid DNA in combination mimic the endogenous IFN-alpha inducer in systemic lupus erythematosus. J Immunol 1999; 163(11):6306-13.
19. Bave U, Alm G V, Ronnblom L. The combination of apoptotic U937 cells and lupus IgG is a potent IFN-alpha inducer. J Immunol 2000; 165(6):3519-26.
20. Bave U, Vallin H, Alm G V, Ronnblom L. Activation of natural interferon-alpha producing cells by apoptotic U937 cells combined with lupus IgG and its regulation by cytokines. J Autoimmun 2001; 17(1):71-80.
21. Bave U, Magnusson M, Eloranta M L, Perers A, Alm G V, Ronnblom L. Fc gamma RIIa is expressed on natural IFN-alpha-producing cells (plasmacytoid dendritic cells) and is required for the IFN-alpha production induced by apoptotic cells combined with lupus IgG. J Immunol 2003; 171(6):3296-302.
22. Lovgren T, Eloranta M L, Bave U, Alm G V, Ronnblom L. Induction of interferon-alpha production in plasmacytoid dendritic cells by immune complexes containing nucleic acid released by necrotic or late apoptotic cells and lupus IgG. Arthritis Rheum 2004; 50(6): 1861-72.
23. Means T K, Latz E, Hayashi F, Murali M R, Golenbock D T, Luster A D. Human lupus autoantibody-DNA complexes activate DC through cooperation of CD32 and TLR9. J Clin Invest 2005; 115:407-17.
24. Sledz C A, Holko M, de Veer M J, Silverman R H, Williams B R. Activation of the interferon system by short-interfering RNAs. Nat Cell Biol 2003; 5(9):834-9.
25. Yoneyama M, Kikuchi M, Natsukawa T, Shinobu N, Inaizumi T, Miyagishi M, et al. The RNA helicase RIG-1 has an essential function in double-stranded RNA-induced innate antiviral responses. Nat Immunol 2004; 5:730-7.
26. Kato H, Sato S, Yoneyma M, Yamamoto M, Uematsu S, Matsui K, et al. Cell type-specific involvement of RIG-1 in antiviral response. Immunity 2005; 23:19-29.
27. Tan E M, Cohen A S, Fries J F, Masi A T, McShane D J, Rothfield N F, et al. The 1982 revised criteria for the classification of systemic lupus erythematosus. Arthritis Rheum 1982; 25(11):1271-7.
28. Arnett F C, Edworthy S M, Bloch D A, McShane D J, Fries J F, Cooper N S, et al. The American Rheumatism Association 1987 revised criteria for the classification of rheumatoid arthritis. Arthritis Rheum 1988; 31(3):315-24.
29. Roman M J, Shanker B A, Davis A, Lockshin M D, Sammaritano L, Simantov R, et al. Prevalence and correlates of accelerated atherosclerosis in systemic lupus erythematosus. N Engl J Med 2003; 349(25):2399-406.
30. Kirou K A, Lee C, Crow M K. Measurement of cytokines in autoimmune disease. Methods Mol Med 2004; 102:129-54.
31. Khabar K S, Al-Haj L, Al-Zoghaibi F, Marie M, Dhalla M, Polyak S J, Williams B R. Expressed gene clusters associated with cellular sensitivity and resistance towards anti-viral and anti-proliferative actions of interferon. J Mol Biol 2004; 342:833-46.
32. Ronnblom L, Alm G V. A pivotal role for the natural interferon alpha-producing cells (plasmacytoid dendritic cells) in the pathogenesis of lupus. J Exp Med 2001; 94:F59-63.
33. Crow M K, Kirou K A. Interferon-alpha in systemic lupus erythematosus. Curr Opin Rheumatol 2004; 16(5):541-7.
34. Cederblad B, Blomberg S, Vallin H, Perers A, Alm G V, Ronnblom L. Patients with systemic lupus erythematosus have reduced numbers of circulating natural interferon-α-producing cells. J Autoimmun 1998; 11:465-70.
35. Blomberg S. Eloranta M L, Cederblad B, Nordlind K, Alm G V, Ronnblom L. Presence of cutaneous interferon-α producing cells in patients with systemic lupus erythematosus. Lupus 2001; 10:484-90.
36. Farkas L, Beiske K, Lund-Johansen G, Brandzaeg P, Jahnsen F L. Plasmacytoid dendritic cells (natural interferon-α/β-producing cells) accumulate in cutaneous lupus erythematosus lesions. Am J Pathol 2001; 159:237-243.
37. Kramer M J, Dennin R, Kramer C, Jones G, Connell E, Rolon N, et al. Cell and virus sensitivity studies with recombinant human alpha interferons. J Interferon Res 1983; 3:425-35.
38. Veckman V, Osterlund P, Fagerlund R, Melen K, Matikainen S, Julkunen I. TNF-alpha and IFN-alpha enhance influenza-A-virus-induced chemokine gene expression in human A549 lung epithelial cells. Virology, 2006; 345(1):96-104.
39. Khabar K S, Al-Haj L, Al-Zoghaibi F, Marie M, Dhalla M, Polyak S J, Williams B R. Expressed gene clusters associated with cellular sensitivity and resistance towards anti-viral and anti-proliferative actions of interferon. J Mol Biol. 2004; 342(3):833-846.
40. Tanaka Y, Marusawa H, Seno H, Matsumoto Y, Ueda Y, Kodama Y, Endo Y, Yamauchi J, Matsumoto T, Takaori-Kondo A, Ikai I, Chiba T. Anti-viral protein APOBEC3G is induced by interferon-alpha stimulation in human hepatocytes. Biochem Biophys Res Commun. 2006; 341(2):314-319.
41. Overall J C Jr, Yeh T J, Kern E R. Activity of human recombinant and lymphoblastoid interferons in human and heterologous cell lines. J Interferon Res. 1984; 4(4):529-33.
42. Kubes M, Fuchsberger N, Kontsek P. Cross-species antiviral and antiproliferative activity of human interferon-omega. J Interferon Res. 1994; 14(2):57-9.
43. Niewold T B, Rivera T L, Hua J, Buyon J P, Crow M K. Type I interferon expression in Ro/La autoantibody positive mothers. American College of Rheumatology, Meeting Abstract, submitted May 2006.
44. Zuker M. Mfold web server for nucleic acid folding and hybridization prediction. Nucleic Acids Res. 2003; 31:3406-3415.
45. Kynriakos A K, Cole P, Salmon J E, Roman M J, Crow M K. Identification of molecular pathways associated with progression of carotid atherosclerosis in systemic lupus erythematosus. American College of Rheumatology, Meeting Abstract, submitted May 2006.
46. Hjelmervik T O, Petersen K, Jonassen I, Jonsson R, Boistad A I. Gene expression profiling of minor salivary glands clearly distinguishes primary Sjogren's syndrome patients from healthy control subjects. Arthritis Rheum. 2005 May; 52(5): 1534-44.
47. Freeman W M, Walker S J, Vrana K E. Quantitative RT-PCR: pitfalls and potential. BioTechniques, 1999; 26(1): 112-125.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, Accession Numbers, Figures, Sequence Listings, product descriptions, and protocols are cited throughout this application, the disclosures of which are incorporated by reference herein in their entireties for all purposes.

What is claimed is:

1. A method for diagnosing systemic lupus erythematosus (SLE) in a subject comprising:
a) contacting interferon responsive cells in vitro with a body fluid sample obtained from the subject, wherein the interferon responsive cells are not the subject's cells;
b) detecting the expression level of at least one interferon-inducible gene (IFIG) by the interferon responsive cells of step a);
c) detecting the expression level of at least one interferon-inducible gene (IFIG) by control interferon responsive cells, wherein the control interferon responsive cells are not the subject's cells; and
d) comparing the expression level detected in step b) with the expression level detected in step c), wherein an increased expression level detected in step b) as compared with the expression level detected in step c) is indicative that the subject has SLE.

2. The method of claim 1, comprising contacting the control interferon responsive cells of step c) with a body fluid sample obtained from a healthy subject, prior to detecting.

3. The method of claim 1, comprising contacting the control interferon responsive cells of step c) with media, prior to detecting.

4. The method of claim 1, wherein the interferon responsive cells are selected from the group consisting of A-549 cells, AG1732 cells, HeLa cells, HepG2 cells, Hep-2 cells, Huh-7 cells, G-361 cells, and WISH cells.

5. The method of claim 1, wherein the interferon responsive cells are WISH cells.

6. The method of claim 1, wherein the detecting steps are carried out using real-time quantitative PCR.

7. The method of claim 1, wherein the interferon-inducible genes (IFIG) are interferon-α inducible genes.

8. The method of claim 7, wherein the interferon-α inducible genes are selected from the group consisting of IF144 (SEQ ID NO:16), C1orf29 (SEQ ID NO:20), PRKR (SEQ ID NO:18), IFIT1 (SEQ ID NO:15), and MX1 (SEQ ID NO:22).

9. The method of claim 1, wherein the body fluid sample is plasma or serum.

10. A method for monitoring a subject with systemic lupus erythematosus (SLE) comprising:
a) contacting interferon responsive cells in vitro with a body fluid sample obtained from a subject prior to administration of a test compound, wherein the interferon responsive cells are not the subject's cells;
b) contacting interferon responsive cells in vitro with a body fluid sample obtained from a subject at one or more time points following administration of a test compound, wherein the interferon responsive cells are not the subject's cells;
c) detecting the expression level of at least one interferon-inducible gene by the interferon responsive cells of step a);
d) detecting the expression level of at least one interferon-inducible gene by the interferon responsive cells of step b); and
e) comparing the expression level of step c) with the expression level of step d), wherein a decreased expression level of at least one interferon-inducible gene by the cells of step d) as compared to the expression level of step c) is indicative of efficacy of the test compound.

11. The method of claim 10, wherein the interferon responsive cells are selected from the group consisting of A-549 cells, AG1732 cells, HeLa cells, HepG2 cells, Hep-2 cells, Huh-7 cells, G-361 cells, and WISH cells.

12. The method of claim 10, wherein the interferon responsive cells are WISH cells.

13. The method of claim 10, wherein the detecting steps are carried out using real-time quantitative PCR.

14. The method of claim 10, wherein the interferon-inducible genes (IFIG) are interferon-α inducible genes.

15. The method of claim 14, wherein the interferon-α inducible genes are selected from the group consisting of IFI44 (SEQ ID NO:16), C1orf29 (SEQ ID NO:20), PRKR (SEQ ID NO:18), IFIT1 (SEQ ID NO:15), and MX1 (SEQ ID NO:22).

16. The method of claim 10, wherein the body fluid sample is plasma or serum.

17. A method for detecting mediators that stimulate expression of interferon-inducible genes in a subject comprising:
a) contacting interferon responsive cells in vitro with a body fluid sample obtained from the subject, wherein the interferon responsive cells are not the subject's cells;
b) detecting the expression level of at least one interferon-indLicible gene (IFIG) by the interferon responsive cells of step a);
c) detecting the expression level of at least one interferon-inducible gene (IFIG) by control interferon responsive cells, wherein the control interferon responsive cells are not the subject's cells; and
d) comparing the expression level detected in step b) with the expression level detected in step c), wherein an increased expression level in step b) is indicative that the subject has an elevated level of mediators that stimulate expression of interferon-inducible genes, and the elevated level is indicative of an autoimmune disorder or disease.

18. The method of claim 17, comprising contacting the control interferon responsive cells of step c) with a body fluid sample obtained from a healthy subject, prior to detecting.

19. The method of claim 17, comprising contacting the control interferon responsive cells of step c) with media, prior to detecting.

20. The method of claim 17, wherein the interferon responsive cells are selected from the group consisting of A-549 cells, AG1732 cells, HeLa cells, HepG2 cells, Hep-2 cells, Huh-7 cells, G-361 cells, and WISH cells.

21. The method of claim 17, wherein the interferon responsive cells are WISH cells.

22. The method of claim 17, wherein the detecting steps are carried out using real-time quantitative PCR.

23. The method of claim 17, wherein the interferon-inducible genes are interferon-α inducible genes.

24. The method of claim 23, wherein the interferon-α inducible genes are selected from the group consisting of IFI44 (SEQ ID NO:16), C1orf29 (SEQ ID NO:20), PRKR (SEQ ID NO:18), IFIT1 (SEQ ID NO:15), and MX1 (SEQ ID NO:22).

25. The method of claim 20, wherein the body fluid sample is plasma or serum.

26. A method for assessing the efficacy of a test compound in treating systemic lupus erythematosus (SLE) by:
a) contacting interferon responsive cells in vitro with a body fluid sample obtained from a subject prior to administration of a test compound, wherein the interferon responsive cells are not the subject's cells;
b) contacting interferon responsive cells in vitro with a body fluid sample obtained from a subject at one or more time points following administration of a test compound, wherein the interferon responsive cells are not the subject's cells;
c) detecting the expression level of at least one interferon-inducible gene by the interferon responsive cells of step a);
d) detecting the expression level of at least one interferon-inducible gene by the interferon responsive cells of step b); and
e) comparing the expression level of step c) with the expression level of step d), wherein a decreased expression level of at least one interferon-inducible gene by the cells of step d) as compared to the expression level of step c) is indicative of efficacy of the test compound.

27. The method of claim 26, wherein the interferon responsive cells are selected from the group consisting of A-549 cells, AG1732 cells, HeLa cells, HepG2 cells, Hep-2 cells, Huh-7 cells, G-361 cells, and WISH cells.

28. The method of claim 26, wherein the interferon responsive cells are WISH cells.

29. The method of claim 26, wherein the detecting steps are carried out using real-time quantitative PCR.

30. The method of claim 26, wherein the interferon-inducible genes are interferon-α inducible genes.

31. The method of claim 30, wherein the interferon-α inducible genes are selected from the group consisting of IF144 (SEQ ID NO:16), C1orf29 (SEQ ID NO:20), PRKR (SEQ ID NO:18), IFIT1 (SEQ ID NO:15), and MX1 (SEQ ID NO:22).

32. The method of claim 26, wherein the body fluid sample is plasma or serum.
